(19) United States
(12) Patent Application Publication
(10) Pub. No.: US 2010/0222877 A1
(43) Pub. Date: Sep. 2, 2010

(54) Tissue Engineered Human Pulmonary Valves with Cyclic Pressure Bioreactor Accelerated Seeding Strategies and Methods For Assessing Inflammatory Potential of Putative Scaffolds for Tissue Engineered Heart Valves

(75) Inventor: Richard Hopkins, Kansas City, MO (US)

Correspondence Address: POLSINELLI SHUGHART PC, 700 W. 47TH STREET, SUITE 1000, KANSAS CITY, MO, 64112-1802, US

(73) Assignee: The Children's Mercy Hospital, Kansas City, MO (US)

(21) Appl. No.: 12/716,253

(22) Filed: Mar. 2, 2010

Related U.S. Application Data
(60) Provisional application No. 61/156,847, filed on Mar. 2, 2009.
(60) Provisional application No. 61/178,485, filed on May 14, 2009.

(51) Int. Cl. A61F 2/24; C12N 5/07

(52) U.S. Cl. 623/2.13; 435/325; 435/395

(57) ABSTRACT

The invention provides for bioengineered or tissue engineered heart valves that are more efficiently recellularized and/or have a decreased inflammatory potential. The heart valves are generally decellularized and then recellularized using autologous cells wherein the valves are subjected to pulsatile motion during the recellularization process. Tissue engineered heart valves subjected to the pulsatile motion are characterized by having at least 20% of the cells that remain on or in said previously decellularized tissue two weeks after the recellularization process are located below or interior to the basement membrane of said tissue. A method of making bioengineered tissues having these characteristic is also disclosed. Further provided is a bio-assay and related method for determining the inflammatory potential of a tissue.

### RELATED APPLICATIONS

This application claims priority to U.S. Provisional Patent Application No. 61/156,847, filed on Mar. 2, 2009, the teaching and contents of which are hereby incorporated by reference.

### BACKGROUND

Numerous types of tissue engineered constructs and vascular grafts have been produced over the last few decades. Previous tissue constructs have included man-made polymers as substitutes for various portions of the organ to which the tissue belongs. Materials such as Teflon and Dacron have been used in various configurations including scaffoldings, tissue engineered blood vessels, and the like. Nanofiber self-assemblies have been used as microscaffolds upon which cells are grown. Textile technologies have been used in the preparation of non-woven meshes made of different polymers. The drawback to these types of technologies is that it is difficult to obtain high porosity and a regular pore size, which contributes to unsuccessful cell seeding. Solvent casting and particulate leaching is a technique that allows for an adequate pore size, but the thickness of the graft is limited. Another disadvantage of this technique is that organic solvents must be used and fully removed to avoid damage to cells seeded on the scaffold. This can be a long and difficult process. Gas foaming, where gas acts as a porogen, has been used to avoid the use of organic solvents. Gas foaming has the disadvantage of requiring unusually high temperatures in order to form the gas pores, thereby prohibiting the incorporation of any temperature labile material into the polymer mix. Additionally, the pores do not form an interconnected structure. Emulsification or freeze-drying and thermally induced phase separation both have the disadvantage of irregular pore size and quality.

Currently approved clinical biological/bioprosthetic heart valve replacement options (allografts and xenografts) often result in reduced durability (likely due to innate inflammation and immune rejection and consequential calcification), ultimately leading to accelerated failure.

Significant drawbacks are present with each available prosthetic valve replacement using current technology, including durability challenges, thrombogenicity and immunogenicity. Further, none have demonstrated the capacity to grow or remodel. What is needed in the art is a tissue-engineered valve comprised of a natural extracellular matrix and seeded cells, which could mitigate many of the limitations of previous valves. Although a number of scaffolds, both biologic and synthetic, have been considered for clinical valve replacement, a decellularized allograft avoids many design and antigenicity difficulties present in previous grafts. Such a scaffold, re-seeded with appropriate autologous cells, could yield a tissue engineered heart valve (TEHV) capable of the growth, and constructive and adaptive remodeling necessary to maintain tissue function for the life of the recipient. It is also desired that the valve be clinically useful, meaning that it would need to be prepared within tolerable time constraints, utilizing readily available cells.

Cryopreserved “viable” (i.e., containing donor cells) homografts as currently used are known to have limited durability due to inflammation and immune rejection resulting in fibrosis and calcification of the implanted valves resulting in valvular stenosis and/or insufficiency. Efficient decellularization can remove antigenic components from donor homograft valves, perhaps providing an antigen devoid of collagen/elastin extracellular matrix (ECM) scaffold that retains optimal structural elements of normal semilunar valves.

Decellularized homografts are clinically attractive as they surgically can be tailored homologously for size and location. Advantageously, they achieve immediate normal function postimplantation. Moreover, if the decellularization effectively removes substantially all and preferably all of the cells, the proinflammatory potential, other than of the non-immune wound healing type, will be greatly reduced or eliminated, thereby increasing the potential for prolonged durability. If such decellularized ECM valve scaffolds are not provocative of inflammation other than of the nonimmune wound healing type, then these may be suitable substrates for tissue engineering of viable valves (TEHVs) using ex vivo cell seeding and/or in vivo recellularization methods.

Foreign materials implanted in the human body may elicit various responses such as acute or subacute inflammation, wound healing, fibrous encapsulation, calcification, degradation, thrombus formation, endothelial hyperplasia and chronic inflammatory cell infiltration with fibrous scarring. These reflect a spectrum of responses to challenges by the innate immune system typically referred to as “foreign body reaction.” Macrophages are central to the activation, propagation and titration of this foreign body reaction. Depending on their source and inherent characteristics, all biomaterials may provoke either or both nonspecific and immune mediated innate inflammation. Such mechanisms have been linked to durability and performance issues with bioprosthetic, allograft and xenograft cardiac valves.

Immune mechanisms of inflammation are recognized as critical to the durability of bioprosthetic, cryopreserved allografts and even native heart valves. Bioprosthetic valves typically fail due to inflammation, fibrosis, and ultimately calcification, as do biological valves such as cryopreserved pulmonary and aortic homografts. Interestingly, autograft pulmonary valves functioning as neoaortic valves rarely, if ever, calcify or fail due to stenosis, but rather by dilatation and aneurysm formation. Homograft (allograft) semilunar valves are attractive as proven design optimal platforms for tissue engineering viable “personal” valves. Completely decellularized allograft valve scaffolds, such as those of the present invention, do not retain HLA or ABO antigenicity and theoretically should not stimulate adaptive immune rejection and, in the absence of mechanical irritations or physical-chemical toxicity, might not significantly provoke the innate or non-specific immune system. In contrast, the retained viable cells in cryopreserved homograft valves are capable of stimulating both innate and adaptive specific immune responses. The latter are likely responsible for the observation of second set rejection causing accelerated allograft reoperations following a first allograft conduit cardiac reconstruction. Proinflammatory stimulation within native aortic valve leaflets involving interstitial cells has been linked to gene expression and protein synthesis of inflammation and calcification promoters, suggesting a mechanistic role in the pathogenesis of degenerative calcific native aortic valve stenosis perhaps analogous to the classic fibro-calcific degeneration of homograft valve conduit transplants. For both native and functional biological heart valve implants, heart valves, the consequences of this sequence are loss of hydraulic performance, hemodynamic dysfunction, excessive ventricular loading (volume, pressure or both), and ultimately surgical replacement.

Although the pathogenesis of valve calcification is multifactorial, the current most likely mechanistic theory as to why manufactured xenograft bioprostheses initially do very well, then ultimately fail, is that as the collagen crosslinking agents (eg, glutaraldehyde) dissipate over time, antigen sites are unmasked leading to immune rejection and inflammation which result in degradation, calcification and materials failure.

Tissue decellularization methods are multiple and variable in efficacy. Retained donor cells, cell debris, or other antigen rich sources could provoke immune responses deleterious to the allograft matrix proteins. If such scaffolds contain only structural proteins, theoretically, within species, these should be minimally provocative, behaving similarly to autologous surgical tissue transfers. Xenogeneic sources might behave differently. Using a nonantigenic ECM scaffold and by using a strategy of seeding with autologous cells, then theoretically, a viable structure could be engineered that provokes minimal foreign body reaction. If so achieved, then by definition, the early signaling steps in the inflammatory cascade choreographed by activated macrophages should be absent or muted demonstrating a “profile” of minimal cytokine signaling.

Nonbiologic materials commonly used in cardiovascular applications and generally felt to be relatively “inert” such as nitinol and PTFE might be exemplary of materials with minimal inflammatory potential, and thus could potentially define a useful scale for identifying implantable materials exhibiting minor or “benign foreign body” responses of the innate immune system. Such responses would be characterized by low intensity and duration of inflammation/rejection; reflected quantitatively at the signaling level where one would postulate, at most, a brief, low level expression of early (upstream) cytokines such as TNF-α or IL-1, which would then rapidly abate.

What is needed in the art are methods for recellularization of tissues that repopulate the cells of the tissue in a more efficient and consistent manner, such that the tissue has a better chance of being successful long-term in the patient after implantation. Further, what is needed is a method for producing bioengineered, specifically, tissue engineered constructs that have a reduced inflammatory response when transplanted into a patient. Further, an assay is need to determine the inflammatory response of tissues prior to implantation, such that longevity of transplanted tissue can be determined. Tissue constructs having these characteristics are also desired. What is further needed is a structural scaffold that has been processed such that proinflammatory responses are reduced or eliminated. What is still further needed are tissue engineered heart valves produced by seeding optimally conditioned scaffolds.

### SUMMARY OF INVENTION

The present invention overcomes problems inherent in the prior art and provides a distinct advance in the state of the art by providing tissues for use in bioengineering and tissue engineering applications that are more efficiently recellularized and have a reduced inflammatory response.

Tissue-based circulating monocytes home to the location of any implanted material and in response to the challenge, differentiate into macrophages which become activated thereby driving the overall foreign body response via the production of inflammatory mediators such as cytokines, chemokines and matrix modifying proteins. While other cell types, such as lymphocytes, play a subsequent direct local as well as paracrine and juxtacrine roles in enhancing adherent macrophage and foreign body giant cell activation, it is the activated macrophage which appears to initially coordinate and modulate the intensity and type of responses. Material dependent differences in macrophage mediated inflammatory gene expression during such foreign body reactions have been previously documented. These cells are stimulated by the specific challenge which calibrates the duration and intensity of immuno-inflammatory responses, as modulated by cytokine signaling, thus providing the rationale for targeting the latter for quantitative assays to assess the inflammatory potential of a specific biomaterial.

The present invention provides for tissue engineered heart valves that are more efficiently recellularized and/or have a reduced inflammatory response. The tissue engineered heart valve of the present invention preferably has at least 5% of seeded cells present below the basement membrane, more preferably at least 10% of the seeded cells, 20% of the seeded cells, more preferably, at least 30% of the seeded cells, even more preferably, at least 40% of the seeded cells, more preferably, at least 50% of the seeded cells, still more preferably, at least 60% of the seeded cells, more preferably, at least 70% of the seeded cells, even more preferably, at least 80% of the seeded cells, still more preferably, at least 90% of seeded cells, and most preferably, at least 95% of seeded cells below the basement membrane after about 2 weeks post-recellularization or post-seeding. Advantageously, by having the seeded cells present below the basement membrane, they are not washed off the tissue surface or are disturbed due to the shear forces and stress of the pulsatile motion within the fluid environment.

The tissue engineered heart valve preferably has a reduced inflammatory potential or provokes a reduced inflammatory response, in comparison to other currently available replacement heart valves or constructs. Preferably, the tissue engineered heart valve is based on or uses a non-inflammatory scaffold. Any non-inflammatory scaffold for tissue engineering applications will work for the purposes of the present invention. Preferably, the scaffold is selected from the group consisting of decellularized allograft valves, decellularized xenograft extracellular matrix ECM valves, biodegradable polymers, or other hybrids with ECM proteins plus polymers. In a most preferred embodiment, the scaffold is a decellularized allograft heart valve. The reduced inflammatory response or potential is determined by the measurement of cytokine expression or the level of cytokine mRNA. The scaffold must be non-inflammatory or have a decreased inflammatory potential, as this will affect the outcome of the inflammatory response of the tissue engineered construct. The measurement of cytokine expression falls into two categories: those measured by amount mRNA produced and those measured by actual protein expression. The cytokines measured by protein expression are preferably selected from the group consisting of IL-β, IL-1ra, IL-2, IL-2, IL-4, IL-5, IL-6, IL-7, IL-8, IL-10, IL-12 (p40), IL-13, IL-15, IL-17, TNF-α, INF-α, INF-γ, GM-CSF, MIP-1α, MIP-1β, IP-10, MIG, Exotaxin RANTES, MCP-1, and combinations thereof. The cytokines preferably measured by amount of mRNA are preferably selected from the group consisting of IL-1β, TNF-α, TGF-β1, INF-γ, IL-2, IL-6, IL-8, IL-10, CCR7, CD68, CD163, CCL1, CCL11, CCL13, CCL15, CCL16, CCL17, CCL18, CCL19, CCL2, CCL3, CCL4, CCL5, CCL7, CCL8, CXCL1, CXCL10, CXCL11, CXCL12, CLCX13, CLCX2, CXCL3, CXCL5, CXCL6, CXCL9, CCR1, CCR2, CCR3, CCR4, CCR5, CCR6, CCR7, CCR8, CCR10, CCRL1, CCRL2, BLR1, CXCR3, CXCR4, CXCR6, XYFIP2, AGTRL1, BDNF, C5, C5AR1 (GPR77), CCBP2, CKLF, CMTM1, CMTM2, CMTM3, CMTM4, CMKLR1, CSF3, CX3CL1, CX3CR1, ECGF1, GDF5, GPR31, GPR77, CPR81, HIF1A, IL13, IL16, IL18, IL1A, 1L4, IL8, IL8RA, LTB4R, MMP2, MMP7, MYD88, NFKB1, SCYE1, SDF2, SLIT2, TCP10, TLR2, TLR4, TNF, TNFRSF1A, TNFSF14, TREM1, BHL, XCL1, XCR1, and combinations thereof. Most preferably, the cytokines are measured by protein expression and are preferably selected from the group consisting of TNF-α, TGF-1-β, IL-6, IL-2, IL-1-β-1, and combinations thereof.

In a preferred embodiment, a reduced or decreased inflammatory response is one where the cytokine expression or amount of mRNA is considered to be low to very low. These values are standardized, as known by those of skill in the art, for each cytokine measured as shown by reference to known materials in the art such as the Quantikine® Assay Kits (R&D Systems®, Minneapolis, Minn.). For TNF-α expression, very low is considered to be expression of less than about 60 pg/mg and low is considered to be from about 60 pg/mg to about 120 pg/mg (See FIG. 8). For TGF-1-β expression, very low is considered to be expression of less than about 110 pg/mg and low is considered to be from about 110 pg/mg to about 410 pg/mg (See FIG. 9). For IL-6 expression, very low is considered to be expression of less than about 25 mg/pg and low is considered to be from about 25 pg/mg to about 40 pg/mg (See FIG. 10). For IL-2 expression, very low is considered to be expression of less than about 160 pg/mg and low is considered to be from about 160 pg/mg to 400 pg/mg (See FIG. 11). For IL-1-β-1 expression, very low is considered to be expression of less than about 18 pg/mg and low is considered to be from about 18 pg/mg to about 28 pg/mg (See FIG. 12). Preferably, the cytokines are measured at one to five different time intervals, preferably at 6 hours, 24 hours, and 48 hours after challenge.

In one aspect, the invention provides for a method of recellularizing or repopulating a decellularized tissue. The method of recellularization generally comprises the step of reintroducing cells to a decellularized tissue in an environment where cyclic pressure induces pulsatile motion within the environment. The pulsatile motion preferably mimics the flow of a system with a beating heart such that the decellularized tissue is conditioned to operate under conditions similar to those within a live biologic system. Advantageously, the method of the present invention causes the cells used to recellularize the tissue to migrate further into the milieu of the tissue, maintain phenotype, and act as a signaling milieu to attract other cells to the tissue after it is implanted in the recipient. Preferably, this results in a recellularized tissue that more closely resembles a native tissue, when compared to other methods of recellularization.

Preferably, the method of the present invention comprises recellularizing or repopulating a decellularized tissue in an environment in which cyclic pressure has been induced. The method of the present invention advantageously provides for a mechanism by which a greater number of cells reach the inner portions of the decellularized tissue, meaning that the cells migrate past the basement membrane, as well as maintaining the cell phenotype, such that the cells that migrated into the decellularized tissue are more likely to differentiate into cells appropriate for the type of decellularized tissue being recellularized, and still more preferably are able to establish populations of the correct type of cells.

Preferably, the cyclic pressure induced in the environment where the decellularized tissue is recellularized does not disrupt or put damaging levels of stress on the cells therein. Even more preferably, the cyclic pressure ranges from about −20 mmHg to 200 mmHg, more preferably, from about −15 mmHg to 150 mmHg, still more preferably, from about −10 mmHg to 100 mmHg, more preferably, from about −8 mmHg to 50 mmHg, even more preferably, from −5 mmHg to 30 mmHg, and most preferably, from −3 mmHg to 10 mmHg. The preferred range for cyclic pressure is one that does not disrupt or put stress on the cells.

In a preferred form, aspect, or embodiment of the present invention, the cyclic pressure is increased or ramped up over time. The cyclic pressure preferably has a sinusoidal like waveform motion. Preferably, the cyclic pressure is increased or ramped at less than 48 hour intervals, more preferably, at less than 36 hour intervals, and most preferably, at about 24 hour intervals. Preferably there are at least 1-10 cyclic pressure cycles, more preferably, at least 1-8 cyclic pressure cycles, even more preferably, at least 1-6 cycles, more preferably about 2-5, and most preferably, about 3 cycles. Preferably, each cycle ramps between a peak pressure or diastolic pressure and a minimum pressure or systolic pressure. Preferably, the diastolic or peak pressure is from about 3 to 120 mmHg, more preferably, from about 3 to 100 mmHg, more preferably, from about 3 to 50 mmHg, and most preferably, from about 3 to 10 mmHg. The systolic or minimum pressure is preferably from about −10 to 80 mmHg, more preferably, from about −10 to 50 mmHg, still more preferably, from about −10 to 30 mmHg, and most preferably, from about −5 to 3 mmHg. As known in the art, between these cycles of peak pressure and minimum pressure, there can potentially be a transient low pressure that ranges from about −5 to −1 mmHg. Preferably, this transient low pressure lasts only briefly, preferably less than 5 minutes, more preferably less than 1 minute.

In a preferred embodiment where there are 5 cycles, the 5 cycles are preferably 3/0 (peak/min) mmHg, 5/1 mmHg, 7/3 mmHg, 7/5 mmHg, and 10/5 mmHg, where each cycle lasts 24 hours, except the final cycle, which preferably lasts until 12 hours prior to implantation of the tissue in the recipient. In a preferred embodiment, where there are 4 cycles, the 4 cycles are preferably 5/3 mmHg, 7/4 mmHg, 20/11 mmHg, and 33/14 mmHg, where each cycle lasts 24 hours, except the final cycle, which preferably lasts until 12 hours prior to implantation of the tissue in the recipient. In a preferred embodiment where there are 3 cycles, the 3 cycles are preferably 3/0 (peak/min) mmHg, 5/3 mmHg, and 7/4 mmHg, where each cycle lasts 24 hours, except the final cycle, which preferably lasts until 12 hours prior to implantation of the tissue in the recipient.

The cells used to recellularize or repopulate the decellularized tissue are preferably those with potential to form the phenotypically correct cells for the decellularized tissue. In a preferred embodiment where the decellularized tissue is a heart valve, the cell type would preferably be selected from the group consisting of autologous differentiated cells, autologous multipotential cells, allogenic differentiated cells, allogenic multipotential cells, xenogenic cells, embryonic stem cells, and circulating progenitor cells. Autologous differentiated and allogenic differentiated cells are preferably selected from the group consisting of valve interstitial cells and cells from a vascular organ or tissue such as artery or vein cells. Autologous multipotent and allogenic multipotent cells are preferably selected from bone marrow, fat, any tissue with resident multipotent cells, umbilical chord cells, and Wharton's Jelly cells. Preferably, the cells are autologous multipotent cells, more preferably the cells are autologous multipotent bone marrow cells, and most preferably the cells are autologous multipotent mesenchymal stromal cells from bone marrow. Those of skill in the art can determine appropriate cell types for various tissue types. Preferably, there are 2.4×10³ to 2.5×10⁹, more preferably, 2.4×10⁴ to 2.5×10⁸, and most preferably, 2.4×10³ to 2.5×10⁷ cells used for recellularization.

In a preferred embodiment, the environment in which the decellularized tissue is recellularized is a bioreactor. Any bioreactor appropriate for the type of decellularized tissue utilized that has the capability of introducing cyclic pressure in a fluid environment will work for purposes of the present invention. It is preferable that the bioreactor has the appropriate monitoring capability to monitor hemodynamic biologic parameters. Preferably any hemodynamic biologic parameter will be able to be monitored by the bioreactor. More preferably, the bioreactor has the ability to monitor the following parameters: temperature, pH, PO₂, PCO₂, cyclic pressure, cyclic flow, and combinations thereof.

The decellularized tissue can be decellularized by any means available for removing cells from a harvest tissue. Preferably, the tissue is decellularized as described in U.S. Patent Application No. 61/258,666, filed on Nov. 6, 2009, the teaching and contents of which are hereby incorporated by reference.

The method of decellularization generally comprises performing the following steps on a harvested tissue: a muscle shelf debridement, an enzyme treatment, a detergent wash, and an organic solvent extraction. In one embodiment, the method generally comprises the steps of reciprocating osmotic shock sequences, a detergent wash, a RNA-DNA extraction, an enzyme treatment, and an organic solvent extraction. In a further embodiment, the method comprises the steps of reciprocating osmotic shock sequences, a first detergent wash, a second reciprocating osmotic shock sequence, a RNA-DNA extraction, an enzyme treatment, a second detergent wash, and an organic solvent extraction. In an additional embodiment, the method comprises reciprocating osmotic shock sequences, a detergent wash, a second reciprocating osmotic shock sequence, a RNA-DNA extraction, a digestion step, an enzyme treatment, a second detergent step, an organic solvent extraction, an ion-exchange detergent residual extraction, and a final organic extraction. In a particularly preferred embodiment, the method further comprises an additional washing step in addition to all of the steps noted above. This additional washing step is preferably performed after the second detergent step, but before the organic solvent extraction.

Preferably, all harvested tissues are harvested and stored according to the American Association of Tissue Banks Standards for Tissue Banking 12^{th} edition, the contents of which are herein incorporated by reference.

The timing of the method can be altered depending on the type of tissue, size of tissue, and other variables. Generally, the method takes about 2-14 days, but the appropriate amount of time can be determined by one of skill in the art. For example, in the case of a pulmonary valve, the method preferably takes about 2-7 days, more preferably, about 3-6 days, and, most preferably, about 3.5 to 4 days. In contrast, an aortic valve preferably takes about 3-9 days, more preferably, about 4-7 days, and, most preferably, about 5 days.

In one aspect of the decellularization method, the reciprocating osmotic shock sequences include the use of a hypertonic salt solution. The sequence for the reciprocating osmotic shock sequences preferably includes treatment of tissue with a hypotonic solution, preferably double deionized water (“ddH₂O”), followed by a treatment of the tissue with a hypertonic salt solution, followed by a second treatment with a hypotonic solution, preferably ddH₂O. In some preferred forms or embodiments, the hypertonic salt solution includes one or more chlorides. In another preferred embodiment, the hypertonic salt solution comprises normal saline, one or more chlorides, a sugar or sugar alcohol, and combinations thereof. Still more preferably, the solution comprising normal saline, one or more chlorides, and a sugar or sugar alcohol will further comprise NaCl in addition to the “one or more chlorides.” Various sugars or sugar alcohols including Mannitol, polysaccharides, polyols, dulcitol, rhamitaol, inositol, xylitol, sorbitol, rharrose, lactose, glucose, galactose, and combinations thereof are appropriate for use in the present invention. In a preferred embodiment, the sugar alcohol, preferably Mannitol, acts as a free-radical scavenger, removing harmful free radicals from the tissue to prevent damage. Any sugar or sugar alcohol having the properties of a free-radical scavenger are preferred for purposes of the present invention. Preferred chlorides are selected from the group consisting of NaCl, MgCl₂, KCl, and combinations thereof. In one preferred embodiment, the sugar is Mannitol. Preferably, the normal saline solution contains NaCl is in an amount of about 0.2% to 5%, even more preferably from about 0.4%, to 4%, still more preferably from about 0.5% to about 3%, even more preferably from about 0.7% to about 2%, still more preferably from about 0.8% to about 1.5%, and most preferably about 0.9%. Preferably, the chloride is present in the hypertonic salt solution in an amount of from about 15 gm to 75 gm. When NaCl is present in the hypertonic salt solution, it is in an amount of from about 10 gm to 30 gm, even more preferably from about 12 gm to 26 gm, still more preferably from about 14 gm to 22 gm, even more preferably from about 16 gm to 19 gm, and most preferably about 18 gm. When MgCl₂ is present in the hypertonic salt solution, it is in an amount of about 0.5 gm to 6 gm, more preferably from about 0.8 gm to about 5 gm, still more preferably from about 1 gm to 4 gm, even more preferably from about 1.4 gm to about 3 gm, still more preferably from about 1.8 gm to about 2.3 gm, and is most preferably about 2.03 gm. When KCl is present in the hypertonic salt solution, it is generally in an amount of about 50 gm to 100 gm, more preferably from about 60 gm to 90 gm, even more preferably from about 68 gm to 80 gm, still more preferably from about 70 gm to 77 gm, and most preferably about 74.3 gm. In a preferred embodiment, a sugar alcohol, preferably Mannitol, is present in the hypertonic salt solution in an amount of from about 50 gm/L to 500 gm/L, more preferably from about 60 gm/L to 400 gm/L, even more preferably from about 75 gm/L to 250 gm/L, more preferably from about 100 gm/L to 200 gm/L, and most preferably about 125 gm/L. Preferably, the reciprocating osmotic shock sequences fracture the cell walls thereby allowing the enzyme and detergent washes to remove cellular debris.

In a preferred aspect of the decellularization method, the detergent wash includes the use of one or more detergents. The detergents can be nonionic, anionic, zwitterionic, detergents for the use of cell lysis, and combinations thereof. Any nonionic detergents can be used in the present invention. Preferred nonionic detergents include, but are not limited to: Chenodeoxycholic acid, Chenodeoxycholic acid sodium salt, Cholic acid, ox or sheep bile, Dehydrocholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, Glycocholic acid sodium salt hydrate, Glycocholic acid sodium salt, Glycolithocholic acid 3-sulfate disodium salt, Glycolithocholic acid ethyl ester, N-Laurolysarcosine sodium salt, N-Laurolysarcosine salt solution, Lithium dodecyl sulfate, Lugol solution, Niaproof 4, Triton, Triton QS-15, Triton QS-44 solution, 1-Octanesulfonic acid sodium salt, Sodium 1-butanesulfonate, Sodium 1-deccanesulfonate, Sodium 1-dodecanesulfonate, Sodium 1-heptanesulfonate anhydrous, Sodium 1-nonanesulfonate, Sodium 1-propanesulfonate monohydrate, Sodium 2-bromoethanesulfonate, Sodium choleate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurochenodeoxycholic acid sodium salt monohydrate, Taurochenodeoxycholic acid sodium salt hydrate, Taurolithocholic acid 3-sulfate disodium salt, Tauroursodeoxycholic acid sodium salt, Triton X-200, Triton XGS-20 solution, Trizma dodecyl sulfate, Ursodeoxycholic acid, and combinations thereof. Any anionic detergent will work for the purposes of the present invention. Preferred anionic detergents for use in the present invention, include, but are not limited to: BigCHAP, Bis (polyethylene glycol bis[imidazoyl carbonyl]), Brij®, Brij® 35, Brij® 56, Brij® 72, Brij® 76, Brij® 92V, Brij® 97, Brij® 58P, Cremophor® EL (Sigma, Aldrich), N-Decanoyl-N-methylglucamine, n-Decyl a-D-glucopyranoside, Decyl b-D-maltopyranoside, n-Dodecyl a-D-maltoside, Heptaethylene glycol monodecyl ether, n-Hexadecyl b-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Methyl-6-O-(N-heptylcarbamoyl)-a-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycolmonooctadecyl ether, Octaethylene glycol monotetradecyl ether, Octyl-b-D-glucopyranoside, Pentaethylene glycol monodecyl ether, Pentaethylene glycol monohexadecyl ether, Pentaethylene glycol monohexyl ether, Pentaethylene glycol monooctadecyl ether, Pentaethylene glycolmonooctyl ether, Polyethylene glycol ether, Polyoxyethylene, Saponin, Span® 20, Span® 40, Span® 60, Span® 65, Span® 80, Span® 85 (Sigma Aldrich), Tergitol, Tetradecyl-b-D-maltoside, Tetraethylene glycol monodecyl ether, Tetraethylene glycol monododecyl ether, Tetraethylene glycol monomonotetradecyl ether, Triton CF-21, Triton CF-32, Triton DF-12, Triton DF-16, Triton GR-5M, Triton X-100, Triton X-102, Triton X-15, Triton X-151, Triton X-207, Triton, TWEEN® (Sigma Aldrich), Tyloxapol, n-Undecyl b-D-glucopyranoside, and combinations thereof. Any zwitterionic detergent will work for purposes of the present invention. Preferred zwitterionic detergents include, but are not limited to the following: CHAPS, CHAPSO, Sulfobetaine 3-10 (SB 3-10), Sulfobetaine 3-12 (SB 3-12), Sulfobetaine 3-14 (SB 3-14), ASB-14, ASB-16, ASB-C8Ø, Non-Detergent Sulfobetaine (ND SB) 201, DDMAB, DDMAU, EMPIGEN BB®Detergent, 30% Solution, Lauryldimethylamine Oxide (LDAO) 30% solution, ZWITTERGENT® 3-08 Detergent, ZWITTERGENT® 3-10 Detergent, ZWITTERGENT® 3-12 Detergent, ZWITTERGENT® 3-14 Detergent, ZWITTERGENT® 3-16 Detergent, and combinations thereof. In a particularly preferred embodiment, a nonionic detergent is used first followed by an anionic or zwitterionic detergent. In a preferred embodiment, the detergents used are Triton X-100 (Triton), N-lauroylsarcosine Sodium Salt Solution (NLS), and combinations thereof. Preferably, the detergent wash has the effect of solubilizing proteins and lysing cells. Generally, the amount detergent(s) is in an amount of about 0.01% to 1% by volume, more preferably from about 0.03% to 0.5%, and more preferably from about 0.04% to 0.6%, and is most preferably is about 0.05%.

Preferably, the RNA-DNA extraction step comprises an enzyme. In another preferred embodiment, the RNA-DNA extraction comprises an enzyme, one or more salts, a base, and combinations thereof. Preferably the enzyme is a recombinant enzyme or endonuclease. Any endonuclease will work with the methods of the present invention In a preferred embodiment, the enzyme is an endonuclease, even more preferably the endonuclease is Benzonase®. The endonuclease, preferably Benzonase®, is preferably present in the extraction in an amount of about 12.5 units, where one unit of Benzonase® is defined as the amount of enzyme that causes a ΔA₂₆₀ of 1.0 in 30 minutes, which corresponds to complete digestion of 37 μg of DNA (Novagen, United States). Preferably the endonuclease used has the property of removing DNA and RNA that is either single stranded, double stranded, linear or circular. Any endonuclease exhibiting similar properties is preferred for purposes of the present invention. Preferably the salt is a chloride, with one particularly preferred chloride being Magnesium chloride. In another preferred embodiment, the Benzonase® is present in a solution of Mg. Preferably the Mg is a 2-10 mM solution of Mg, and is most preferably about an 8 mM solution. The base is preferably a weak base, more preferably a hydroxide, and, even more preferably, ammonium hydroxide. In one preferred embodiment, the weak base, preferably ammonium hydroxide, is present in an amount from about 5 ul to about 40 ul, even more preferably from about 10 ul to about 30 ul, still more preferably from about 15 ul to about 22 ul, and is most preferably about 20 ul. Preferably, the RNA-DNA extraction has the effect of avoiding antigenicity issues and allowing for enzyme ingestion.

Preferably, the enzyme treatment step includes the use of a recombinant enzyme. The recombinant enzyme is preferably Benzonase®. Preferably, the enzyme treatment avoids antigenicity issues.

In another aspect of the decellularization method, the organic solvent extraction step comprises an alcohol. The alcohol used can be any alcohol, and preferred alcohols are selected from, but are not limited to, the following group: ethyl alcohol, methyl alcohol, n-propyl alcohol, iso-propyl alcohol, n-butyl alcohol, sec-butyl alcohol, t-butyl alcohol, iso-amyl alcohol, n-decyl alcohol and combinations thereof. In one preferred embodiment, the alcohol has a high concentration, preferably higher than 140 proof, even more preferably higher than 160 proof, still more preferably higher than 180 proof, and is most preferably about 200 proof. In preferred forms, the alcohol also acts an anti-calcification agent, one such preferred alcohol is ethyl alcohol. In another preferred embodiment, the organic solvent extraction step includes an ion-exchange detergent residual extraction. The ion-exchange detergent residual extraction preferably comprises microcarrier beads in an open reaction chamber where fluid is continually exchanged throughout the open reaction chamber. Preferably, the beads used in the ion-exchange detergent residual extraction are such that no residual beads are left on the tissue therefore minimizing bead-to-bead interaction. In one preferred embodiment, the extraction has the effect of sterilizing and disinfecting the valve, as well as removing lipids and other hydrophilic residuals. Preferably, the extraction step also has anti-calcification effects.

Preferably, the organic extraction step comprises a salt. More preferably the organic extraction comprises a salt, a saline solution, and water. Even more preferably, the organic extraction comprises a salt, a saline-sugar solution, and water. Preferably the salt is a chloride. In a preferred embodiment, the chloride is selected from the group consisting of NaCl, MgCl₂, KCl, and combinations thereof. Preferably the chloride is MgCl₂. In one preferred embodiment, the saline-sugar solution includes normal saline and a sugar alcohol. Preferably the sugar alcohol is selected from, but not limited to, the following: Glycol, Glycerol, Erythritol, Threitol, Arabitol, Cylitol, Ribitol, Sorbitol, Mannitol, Dulcitol, Iditol, Isomalt, Maltitol, and combinations thereof. Preferably, the sugar alcohol is Mannitol. Preferably, the organic extraction step has the effect of removing the extra water from the interstitium of the tissue reducing the “softening” effects and firming the tissue for safer handling and for better suturing, handling, and surgical characteristics.

The decellularized tissue can come from any source, including, but not limited to, mammals and avian species, more preferably, dogs (canine), cats (feline), sheep (ovine), cows (bovine), pigs (porcine), horses (equine), monkeys (primates), mice, birds, or humans. Preferred tissues include, but are not limited to, vascular tissue, cardiac tissue, and muscle tissue. In a preferred embodiment, the tissue is a human or autologous or mammalian heart valve.

The present invention provides several advantages. The method of the present invention, by using pulsatile motion when recellularizing a decellularized tissue, allows the cells to migrate further into the tissue, when compared to those tissues recellularized using conventional or static recellularization. When tissues are recellularized using pulsatile motion, there is greater consistency of repopulation or distribution of repopulated cells within the tissue than with tissues recellularized using static or conventional methods such that the recellularized tissue of the present invention appears more like native tissues that have not been decellularized. For example, in a heart valve, it was surprisingly found that a greater number of the leaflets repopulated with cells in a more consistent manner than in a heart valve recellularized using static recellularization. In other words, pulsatile recellularization in accordance with the present application results in a repopulation of cells that are distributed more evenly throughout the tissue as compared to the cell repopulation using static recellularization methodologies where the vast majority of cell repopulation is located closer to the surface of the tissue. Further, a greater number of cells remain phenotypically correct, such that a greater number differentiate into tissue-specific cells, when compared to the cells used to recellularize tissues using static recellularization.

In another aspect of the present invention, a method for producing recellularized tissue that has a decreased inflammatory response is provided. It was surprisingly discovered that specific tuning of bioactive materials has the demonstrated potential for attenuating proinflammatory cytokine expression by macrophages. Alternatives for valve scaffolds include: decellularized allograft valves, decellularized xenograft extracellular matrix ECM valves, biodegradable polymers, or hybrids with ECM proteins plus polymers. Because of the risk of leaving in-situ residual necrotic cell debris, incomplete decellularization may be associated with significant activation of proinflammatory and pro-thrombotic cascades. Such effects may be exacerbated by flow related or mechanical effects caused by rough exposed collagen fibers.

The method preferably comprises the steps of obtaining a harvested tissue, decellularizing the tissue and recellularizing the tissue using a bioreactor with pulsatile motion. Preferably, the decellularization process comprises a muscle shelf debridement, an enzyme treatment, a detergent wash, and an organic solvent extraction; and, more preferably, the decellularization process comprises the method comprises reciprocating osmotic shock sequences, a detergent wash, a second reciprocating osmotic shock sequence, a RNA-DNA extraction, a digestion step, an enzyme treatment, a second detergent step, an organic solvent extraction, an ion-exchange detergent residual extraction, and a final organic extraction.

Preferably, a decreased inflammatory response is measured by a reduction in cytokine protein expression or a reduction in the level of cytokine mRNA, when compared to other bio engineered constructs. The measurement of cytokines fall into two categories: those measured by mRNA and those measured by protein expression. The cytokines measured by protein expression are preferably selected from the group consisting of IL-β, IL-1ra, IL-2, IL-2, IL-4, IL-5, IL-6, IL-7, IL-8, IL-10, IL-12 (p40), IL-13, IL-15, IL-17, TNF-α, INF-α, INF-γ, GM-CSF, MIP-1α, MIP-1β, IP-10, MIG, Exotaxin RANTES, MCP-1, and combinations thereof. The cytokines preferably measured by mRNA are preferably selected from the group consisting of IL-1β, TNF-α, TGF-β1, INF-γ, IL-2, IL-6, IL-8, IL-10, CCR7, CD68, CD163, CCL1, CCL11, CCL13, CCL15, CCL16, CCL17, CCL18, CCL19, CCL2, CCL3, CCL4, CCL5, CCL7, CCL8, CXCL1, CXCL10, CXCL11, CXCL12, CLCX13, CLCX2, CXCL3, CXCL5, CXCL6, CXCL9, CCR1, CCR2, CCR3, CCR4, CCR5, CCR6, CCR7, CCR8, CCR10, CCRL1, CCRL2, BLR1, CXCR3, CXCR4, CXCR6, XYFIP2, AGTRL1, BDNF, C5, C5AR1 (GPR77), CCBP2, CKLF, CMTM1, CMTM2, CMTM3, CMTM4, CMKLR1, CSF3, CX3CL1, CX3CR1, ECGF1, GDF5, GPR31, GPR77, CPR81, HIF1A, IL13, IL16, IL18, IL1A, IL4, IL8, IL8RA, LTB4R, MMP2, MMP7, MYD88, NFKB1, SCYE1, SDF2, SLIT2, TCP10, TLR2, TLR4, TNF, TNFRSF1A, TNFSF14, TREM1, BHL, XCL1, XCR1, and combinations thereof. Most preferably, the cytokines are measured by protein expression and are selected from the group consisting of TNF-α, TGF-1-β, IL-6, IL-2, IL-1-β-1, and combinations thereof.

In a preferred embodiment, a reduced or decreased inflammatory response is one where the cytokine expression or amount of mRNA is considered to be low to very low according to standards established in the art for each specific cytokine. These values can be determined by one of skill in the art for each cytokine measured. For TNF-α expression, very low is considered to be expression of less than about 60 pg/mg and low is considered to be from about 60 pg/mg to about 120 pg/mg (See FIG. 8). For TGF-1-β expression, very low is considered to be expression of less than about 110 pg/mg and low is considered to be from about 110 pg/mg to about 410 pg/mg (See FIG. 9) For IL-6 expression, very low is considered to be expression of less than about 25 mg/pg and low is considered to be from about 25 pg/mg to about 40 pg/mg (See FIG. 10). For IL-2 expression, very low is considered to be expression of less than about 160 pg/mg and low is considered to be from about 160 pg/mg to 400 pg/mg (See FIG. 11). For IL-1-β-1 expression, very low is considered to be expression of less than about 18 pg/mg and low is considered to be from about 18 pg/mg to about 28 pg/mg (See FIG. 12). Preferably, the cytokines are measured at one to five different time intervals, more preferably at 3 time intervals. The time intervals, in an embodiment where there are three, are preferably at 6 hours, 24 hours, and 48 hours after challenge.

In yet another aspect of the present invention, a quantitative bio-assay is provided for evaluating the inflammatory potential of tissues utilized as scaffolds for tissue-engineering applications. Preferably, the bio-assay measures the level of cytokines present in a tissue used for a scaffold, bio-engineering application, or tissue-engineering application. Preferably, the assay measures acute phase human-macrophage-centric inflammatory cytokine signaling, when the presence of a foreign body would initially be detected. The bio-assay preferably takes a sampling of cells from a tissue, preferably, an aortic valve, more preferably, a human aortic valve, and measures the level of cytokine expression at 6 hours, 24 hours, and 48 hours after challenge. The cytokines are measured using ELISA for each cytokine measured.

A measurement of cytokine expression that falls in the very low or low parameters is considered a positive result, meaning that the tissue has a decreased or reduced inflammatory response or decreased or reduced inflammatory potential. Preferably, a decreased inflammatory response is measured by a reduction in cytokine protein expression. The measurement of cytokines fall into two categories: those measured by the amount of mRNA and those measured by protein expression. The cytokines measured by protein expression are preferably selected from the group consisting of IL-β, IL-1ra, IL-2, IL-2, IL-4, IL-5, IL-6, IL-7, IL-8, IL-10, IL-12 (p40), IL-13, IL-15, IL-17, TNF-α, INF-α, INF-γ, GM-CSF, MIP-1α, MIP-1β, IP-10, MIG, Exotaxin RANTES, MCP-1, and combinations thereof. The cytokines preferably measured by the amount mRNA are preferably selected from the group consisting of IL-1β, TNF-+, TGF-β1, INF-γ, IL-2, IL-6, IL-8, IL-10, CCR7, CD68, CD163, CCL1, CCL11, CCL13, CCL15, CCL16, CCL17, CCL18, CCL19, CCL2, CCL3, CCL4, CCL5, CCL7, CCL8, CXCL1, CXCL10, CXCL11, CXCL12, CLCX13, CLCX2, CXCL3, CXCL5, CXCL6, CXCL9, CCR1, CCR2, CCR3, CCR4, CCR5, CCR6, CCR7, CCR8, CCR10, CCRL1, CCRL2, BLR1, CXCR3, CXCR4, CXCR6, XYFIP2, AGTRL1, BDNF, C5, C5AR1 (GPR77), CCBP2, CKLF, CMTM1, CMTM2, CMTM3, CMTM4, CMKLR1, CSF3, CX3CL1, CX3CR1, ECGF1, GDF5, GPR31, GPR77, CPR81, HIF1A, IL13, IL16, IL18, IL1A, IL4, IL8, IL8RA, LTB4R, MMP2, MMP7, MYD88, NFKB1, SCYE1, SDF2, SLIT2, TCP10, TLR2, TLR4, TNF, TNFRSF1A, TNFSF14, TREM1, BHL, XCL1, XCR1, and combinations thereof. Most preferably, the cytokines measured by protein expression are TNF-α, TGF-1-β, IL-6, IL-2, IL-1-β-1, and combinations thereof.

In a preferred embodiment, a reduced or decreased inflammatory response is one where the cytokine expression or amount of mRNA is considered to be low to very low. These values can be determined by one of skill in the art. For TNF-α expression, very low is considered to be expression of less than about 60 pg/mg and low is considered to be from about 60 pg/mg to about 120 pg/mg (See FIG. 8). For TGF-1-β expression, very low is considered to be expression of less than about 110 pg/mg and low is considered to be from about 110 pg/mg to about 410 pg/mg (See FIG. 9). For IL-6 expression, very low is considered to be expression of less than about 25 mg/pg and low is considered to be from about 25 pg/mg to about 40 pg/mg (See FIG. 10). For IL-2 expression, very low is considered to be expression of less than about 160 pg/mg and low is considered to be from about 160 pg/mg to 400 pg/mg (See FIG. 11). For IL-1-β-1 expression, very low is considered to be expression of less than about 18 pg/mg and low is considered to be from about 18 pg/mg to about 28 pg/mg (See FIG. 12). Preferably, the cytokines are measured at one to five different time intervals, more preferably at 3 different time intervals. In a preferred embodiment, where there are three time intervals, the three time intervals are preferably at 6 hours, 24 hours, and 48 hours after challenge.

In a further aspect of the present invention, a tissue engineered heart valve comprising a previously decellularized tissue that has undergone a cell seeding process is provided. The TEHV functions as a valve, but has cell-based biologic properties of tissue renewal. Preferably the TEHV is based on a collagen/elastin scaffold derived from allogeneic heart valves. Still more preferably, such a TEHV is imbued with the capacity for structural and adaptive remodeling wherein the tissue is capable of ongoing regeneration as well as responding to changing physiological conditions. Thus, a preferred TEHV of the present invention can reestablish both cellular and noncellular tissue components as well as remodel in response to growth and changing environmental cues. Preferably, the safety margins and functional performance of TEHVs in accordance with the present application are based on optimal designs and experience no degradation of essential properties even prior to complete recellularization. Still more preferably, the in vitro recellularization process only needs to be partially completed in order to establish optimal conditions for effective in vivo cell repopulation (i.e. tissue maturation post implantation). In even more preferred forms, the bioengineered construct produced herein can completely recellularize in vivo due when undergoing the decellularization process described herein. This is because the bioengineered construct or scaffold has been optimally prepared to become a living tissue by using the methods described herein. More preferably, the scaffold is non-inflammatory as measured by cytokine expression. Preferably and advantageously, the tissue engineered heart valve of the present invention is characterized by having at least 20% of the cells that remain on or in said previously decellularized tissue two weeks after the cell seeding process are located below or interior to the basement membrane of said tissue. Even more preferably, the cells below or interior to the basement membrane are substantially evenly distributed throughout the tissue. Still more preferably, at least some, preferably at least 10%, more preferably at least 20%, still more preferably at least 30%, and most preferably at least 40% of the cells that are below the basement membrane are located past the flexion point of the leaflet.

### DEFINITIONS

“Decellularization”, for purposes of the present invention, refers to the process of removing cells and/or cellular debris from a tissue. In a preferred embodiment the decellularization process prepares tissue, such that it is available to accept new cells into its biological scaffold.

“Recellularization”, for purposes of the present invention, refers to the process of repopulating at least a portion of a tissue, scaffold, or other bioengineered construct with cells.

“Cyclic Pressure”, is pressure, or the amount of force acting on a unit area, wherein the pressure has a sinusoidal like waveform motion. Thus, in a fluid environment, cyclic pressure would cause pulsatile motion within the fluid environment.

“Pulsatile Motion”, as used herein, pulsatile motion is a motion that acts as a throbbing or beating, as in the way a heart throbs or beats. The motion provides pulses of motion rather than continuous steady flow or pressure.

“Bioreactor” any device or system that supports a biologically active environment in which cells may remain viable and grow. Preferably, a bioreactor is a vessel in which a process is carried out that involves tissue in a fluid environment with the vessel. Preferably, the bioreactor has tunability (or control over certain parameters) of, but not limited to, temperature, pH, PO₂, PCO₂, cyclic pressure, cyclic flow, and combinations thereof.

“Reduced or Decreased Inflammatory Response”, for purposes of the present invention, refers to a cytokine expression level which has decreased in level of expression or is reduced, in comparison to a cytokine expression level response to the challenge of another tissue exposure in the test chamber. A tissue would be considered to have a reduced inflammatory response when the level of expression is categorized as low to very low for the specific cytokine. It may be correlated with explant pathological evaluation of implants that do not incite as much inflammation and scarring as other known materials.

“Phenotype” or “Phenotypically correct cells”, as used herein, refers to the observable characteristic of a cell, such a morphology, development, biochemical, physiological, or behavioral properties. A phenotypically correct cell exhibits the phenotype appropriate for the type of tissue in which the cell is located and location of the cell with the tissue. A phenotypically correct cell is or can would differentiate into a cell that has the characteristics of a specific cell desired found in native tissue.

“Fluid Environment”, refers to an environment in which movement can be introduced. Preferably, it is a liquid environment.

“Cytokine Protein Expression”, refers to the level of cytokine proteins that are expressed by a cell or cells within a tissue. Preferably, the cytokine protein expression refers to the expression of a cytokine used to measure inflammatory response.

“Inflammatory potential” The proclivity for inciting inflammation characteristic of a specific material or substance as defined by clinical experience, bioassays or surrogate marker testing with methods such as the human macrophage cytokine signaling assay.

“Inflammatory response”, refers to the complex biological responses of vascular tissues to harmful stimuli, such as pathogens, damaged cells, or irritants. Preferably, the inflammatory response, for purposes of the present invention, is measured by the level of cytokine expression in a cell or cells within a tissue.

A “bio engineered” construct, for purposes of the present invention, refers to a construct that provides a surface for a living component to be incorporated therein or thereon. Bio engineered constructs can include scaffolds that are natural or synthetic, as well as seeded scaffolds, referred to as tissue engineered constructs, or in the context of this invention as tissue engineered heart valves. Bio engineered constructs are preferably selected from the group consisting of those made using polymers; extra cellular matrix; manufactured, synthesized, or harvested from an animal donor; extra cellular matrix/polymer hybrids; natural extra cellular matrix; cryopreserved valves; or native tissue constructs. The bioengineered constructs of the present invention are designed to attract cells for repopulation or seeding.

A “tissue engineered” construct, for purposes of the present invention, refers to a construct that incorporates living cells. A tissue engineered construct is a category of bio engineered constructs where the scaffold has been repopulated with tissue appropriate cells.

“Native” tissue or heart valve, refers to tissue that is harvested from a living being.

“Freeze fractured” tissue or heart valve refers to a preparation method where the fresh tissue or cell suspension is frozen rapidly (cryofixed) then fractured by simply breaking or by using a microtome while maintained at liquid nitrogen temperature.

“Debridement”, as used herein, encompasses enzymatic debridement by which dead, contaminated or adherent tissue or foreign materials are removed from a tissue.

“Enzyme treatment”, as used herein, refers to the addition of an enzyme to a solution or treatment of a material, such as tissue, with an enzyme.

“Detergent Wash”, as used herein, refers to the rinsing of a tissue or solution with a detergent. The detergent can be any type of detergent including, but not limited to, nonionic, anionic, detergents for the use of cell lysis, and combinations thereof.

“Solvent Extraction”, as used herein, refers to the separation of materials of different chemical types and solubilities by selective solvent action, that is some materials are more suitable in one solvent than in another, hence there is a preferential extractive action. This process can be used to refine products, chemicals, etc.

“Osmotic Shock” as used herein, is a sudden change in the solute concentration around a cell causing rapid change in the movement of water across the cell membrane. This is possible under conditions of high concentrations of salts, substrates, or any solute in the supernatant causing water to be drawn out of the cells via osmosis. This process inhibits the transport of substrates and cofactors into the cell, thus, “shocking” them.

“Organic Extraction”, for purposes of the present invention, refers to the “solvent extraction” described above, wherein said solvent is of organic nature.

### DESCRIPTION OF FIGURES

FIG. 1: Standard curve for MTT viability assay based on a 7-fold serial dilution with common factor 2. Maximum value is 500,000 cells. R² value represents 4-parametric regression. Values are represented as mean (n=3)±STD;

FIG. 2: Human aortic valve leaflet interstitial cells showing myofibroblast phenotype;

FIG. 3A: Cells seeded onto leaflet per surface area in pulsatile, cyclic pressure culture;

FIG. 3B: Cells seeded onto leaflet per surface area in static culture;

FIG. 4A: Cells seeded onto sinus per surface area in pulsatile, cyclic pressure culture;

FIG. 4B: Cells seeded onto sinus per surface area in static culture;

FIG. 5: Immunohistochemical staining of leaflet and sinus tissue for 5 days at the highest dose;

FIG. 6A: cells remaining in sinus after pulsatile, cyclic pressure culture per cells initially attached, normalized to surface area;

FIG. 6B: cells remaining in leaflet after pulsatile, cyclic pressure culture per cells initially attached, normalized to surface area;

FIG. 7: CD-68 positive staining (red) confirms macrophage differentiation following PMA 400×;

FIG. 8: TNF-α titers at all three times for all materials tested;

FIG. 9: T6F-β1 titers at all three times for all materials (sinus wall and leaflets);

FIG. 10: IL-6 titers at all three times for all materials;

FIG. 11: IL-2 titers at all three time points for all materials tested;

FIG. 12: IL-1β1 titers at all three time points for all materials tested;

FIG. 13: Relative cytokine expressions by human macrophages after six hours of exposure to test materials (only controls and leaflets displayed for clarity);

FIG. 14: Relative cytokine expressions by human macrophages after 24 hours of exposure to test materials (only controls and leaflets displayed for clarity);

FIG. 15: Relative cytokine expression by human macrophages after 48 hours of exposure to test materials (only controls and leaflets displayed for clarity);

FIG. 16: Photograph of a decellularized valve that has not been recellularized or implanted;

FIG. 17: Photograph of a pulmonary artery sinus wall decelled and conditioned at 10 weeks post implant in sheep;

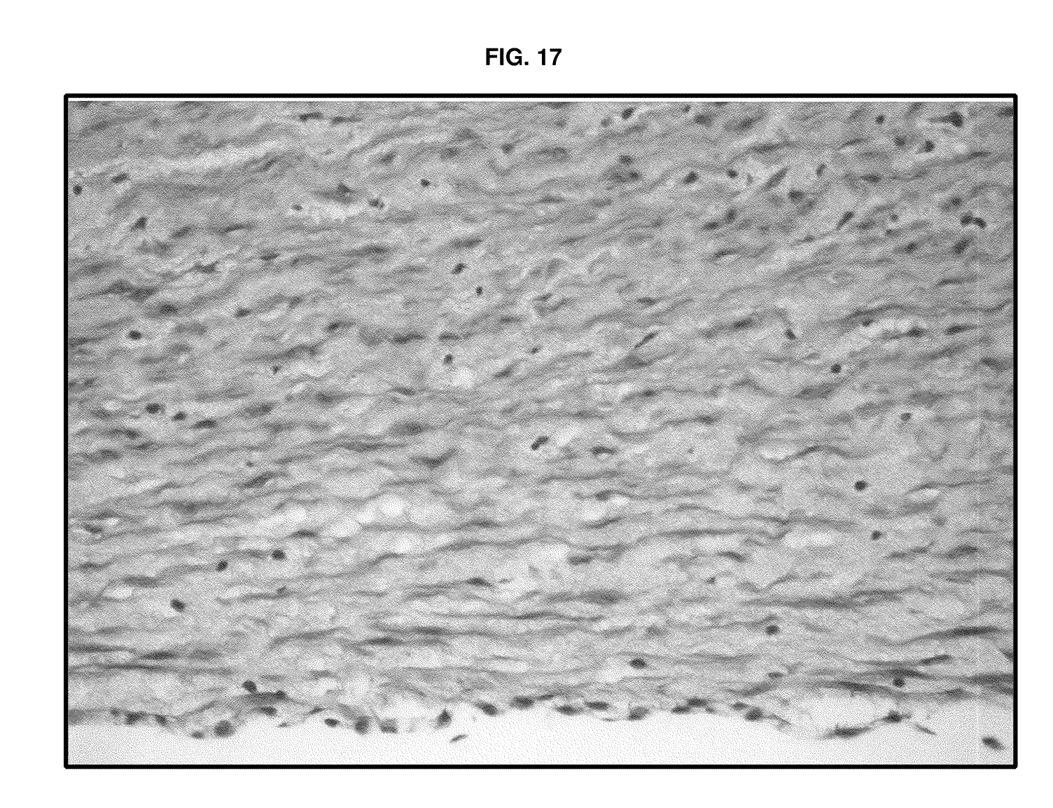

FIG. 18: Photograph of a heart valve that has been decellularized only (no conditioning) at 20 weeks after implant in a sheep;

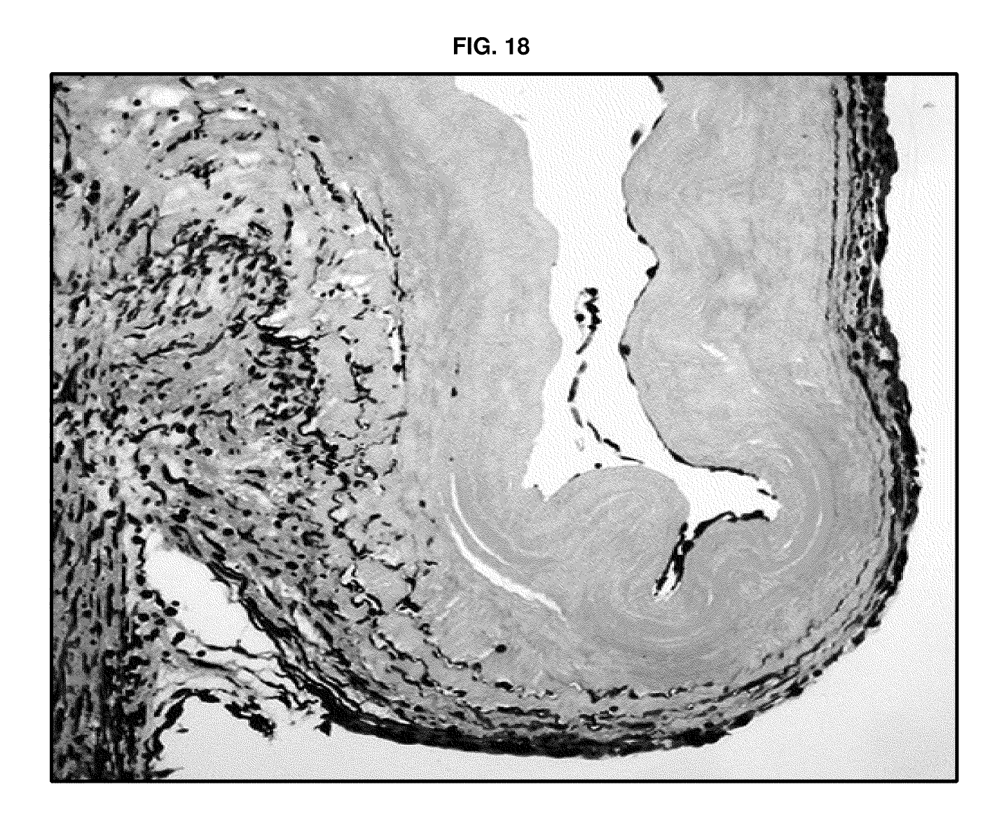

FIG. 19: Photograph of a heart valve after pulsatile seeding of conditional ovine pulmonary valve leaflet;

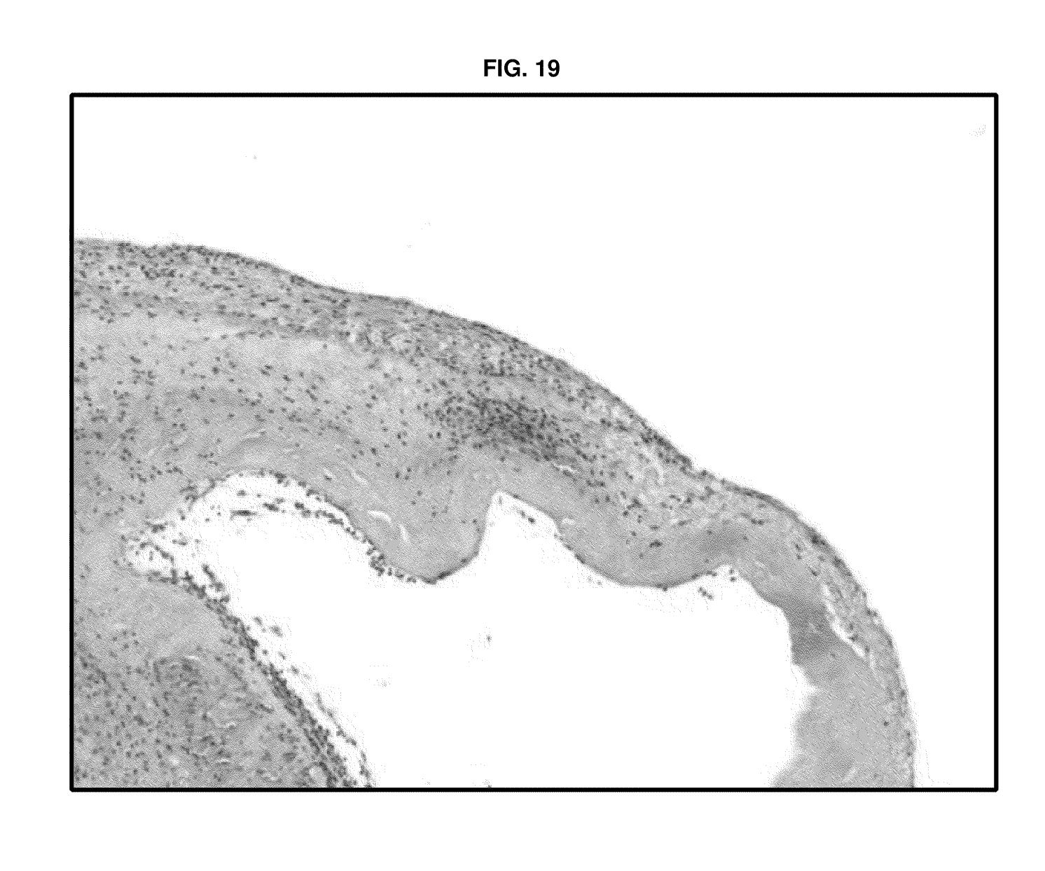

FIG. 20: Photograph of normal native leaflet fresh;

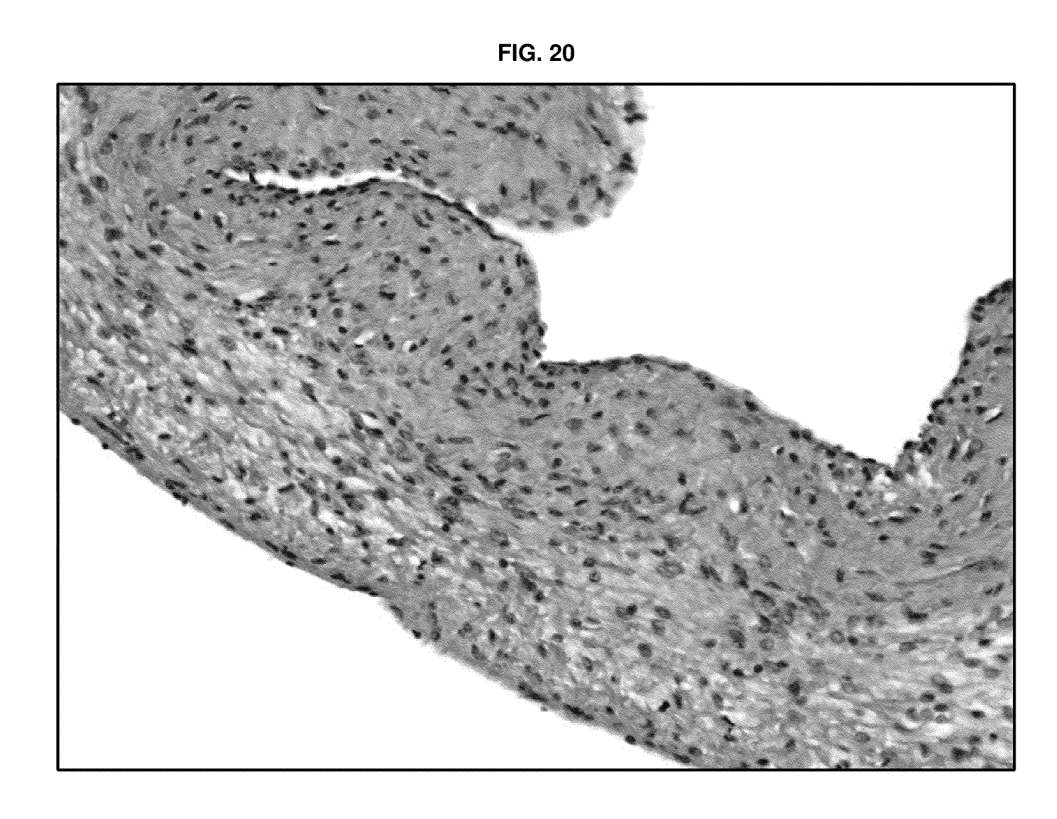

FIG. 21: Photograph of a heart valve after static seeding; and

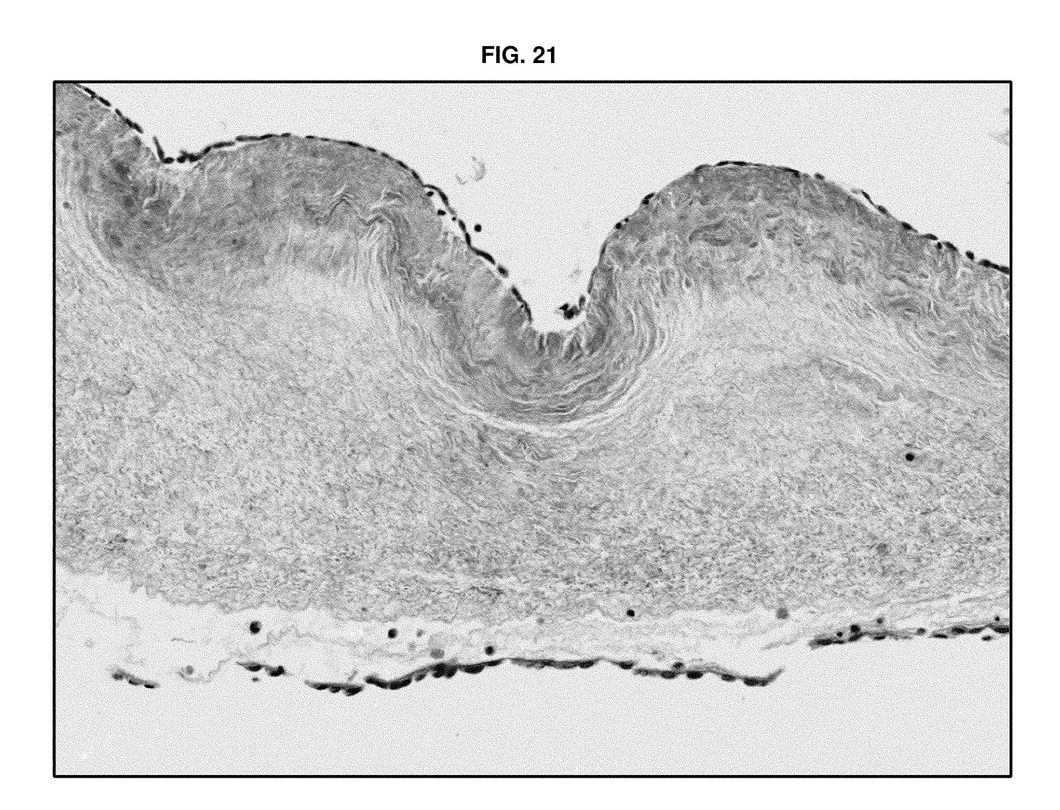

FIG. 22: Photograph of a heart valve recellularized using pulsatile seeding at 52 weeks post implant.

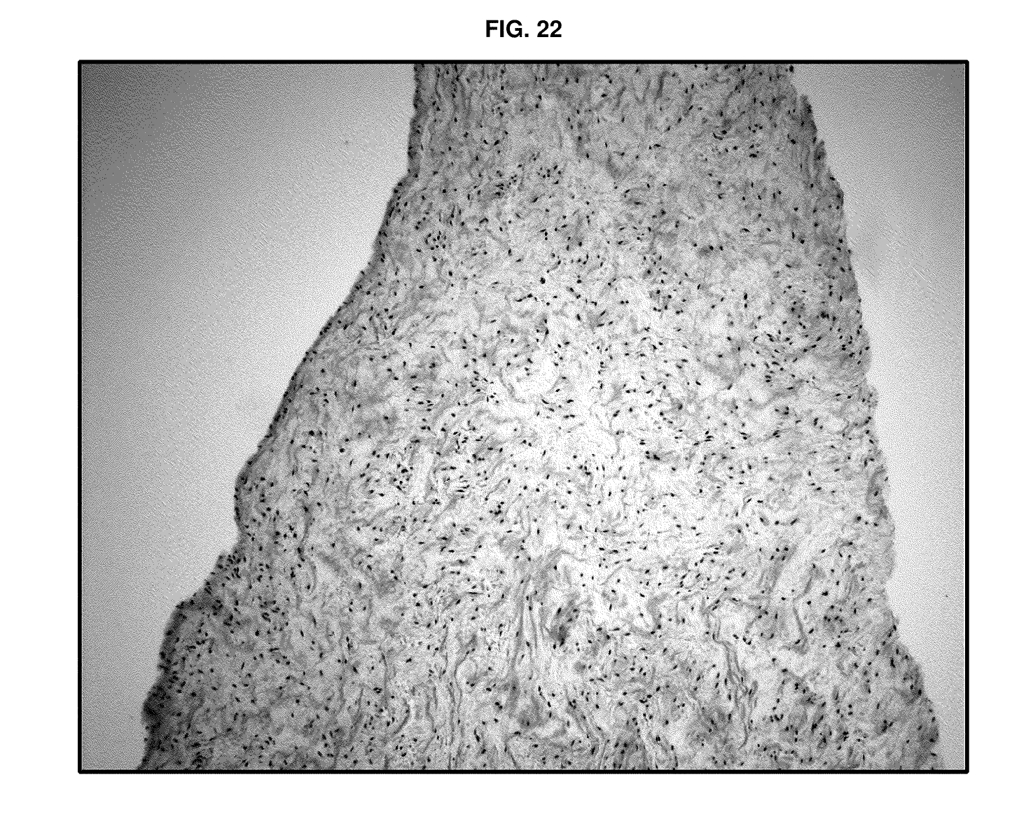

### DETAILED DESCRIPTION

The following examples are representative of preferred embodiments of the present invention. It is understood that nothing herein should be taken as a limitation upon the overall invention.

### Example 1

Significant drawbacks are present with each available prosthetic valve replacement including durability challenges, thrombogenicity, immunogenicity, and of course, surgically related risks. Further, none have demonstrated the capacity to grow or remodel. A tissue-engineered valve comprised of an extracellular matrix and seeded cells could mitigate many of these limitations. Although a number of scaffolds, both biologic and synthetic, have been considered for clinical valve replacement, a decellularized allograft avoids many design and antigenicity difficulties. Such a scaffold, re-seeded with appropriate autologous cells, could yield a tissue engineered heart valve (TEHV) capable of the growth, constructive and adaptive remodeling necessary to maintain tissue function for the life of the recipient. To be clinically useful, such a valve would need to be prepared within tolerable time constraints, utilizing readily available cells.

### Materials and Methods

Human aortic valve leaflet interestitial cells (hVICs) were isolated from cryopreserved aortic valve leaflets using 0.25% trypsin and two iterations of cell scraping. Population purity was confirmed after two passages using an immunocytochemical battery with fluorescent labeling.

Human pulmonary cryopreserved valves were thawed using continuous flow sterile fluid. Intact valves were decellularized using a novel, multi-solvent, reciprocating osmolarity, double detergent, enzyme catalyzed protocol. Complete decellularization was confirmed using H+E, Movat's pentachrome and DAPI nuclear stains.

Leaflets and sinuses were surgically resected from decellularized human pulmonary valves. The sinus was defined as the region of artery wall between the cusp base and sinotubular junction. Each was divided into 5 mm×5 mm pieces for separate assay. N=8 individual biopsies per cell density were transferred into inserts of HTS Transwell-24 well plates. Biopsies were seeded at one of three cell densities under static conditions for 24 hours. Static controls were maintained in well plates for a total of 5 days. Pulsatile samples were transferred at 24 hours to a novel cyclic pressure bioreactor with adjustable peak pressure for four additional days.

When biopsy timepoint was reached, n=6 biopsies were taken for MTT cell viability quantification assay. N=2 biopsies were taken for histological and immunohistochemical analysis. For comparison, two clinically available, manufactured vascular patch scaffolds (Photo-oxidized bovine pericardium, expanded polytetrafluoroethylene) were seeded using the protocol described above.

For all variables, descriptive statistics (Means and Standard Deviations for continuous variables, proportions for categorical variables) were computed. Scaffold types, seeding methods and dose response curves were compared using single factor analysis of variance and a post-hoc Tukey test. A general linear regression model was used for repeated measures (eg, multiple time points). SPSS v.15.0 for Windows Statistical Package was utilized. P<0.05 were considered statistically significant.

### Results and Discussion

TABLE 1

| | a. Static | b. Pulsatile |
|---|---|---|
| c. Leaflet | 894 ± 84 | 80 ± 12 |
| d. Sinus | 838 ± 50 | 79 ± 12 |
| e. Pericardium | 253 ± 16 | 117 ± 10 |
| ePTFE | 64 ± 11 | 43 ± 1 |

Table 1: 4.3 × 10³ cells/mm² (2.5 × 10⁵ cells total) initially seeded and incubated 120 hours in pulsatile, pressurized bioreactors. Final cell numbers represented as cells/mm² (n = 6) SEM.

No statistical difference between leaflet and sinus scaffolds. Seeding methods yielded different cell numbers for leaflet and sinus (p<0.0001), but not for Pericardium or ePTFE. Low and high dose samples showed similar trends (data not shown). Pericardium showed less immigration than sinus and leaflet scaffolds while ePTFE scaffolds allowed neither attachment nor migration.

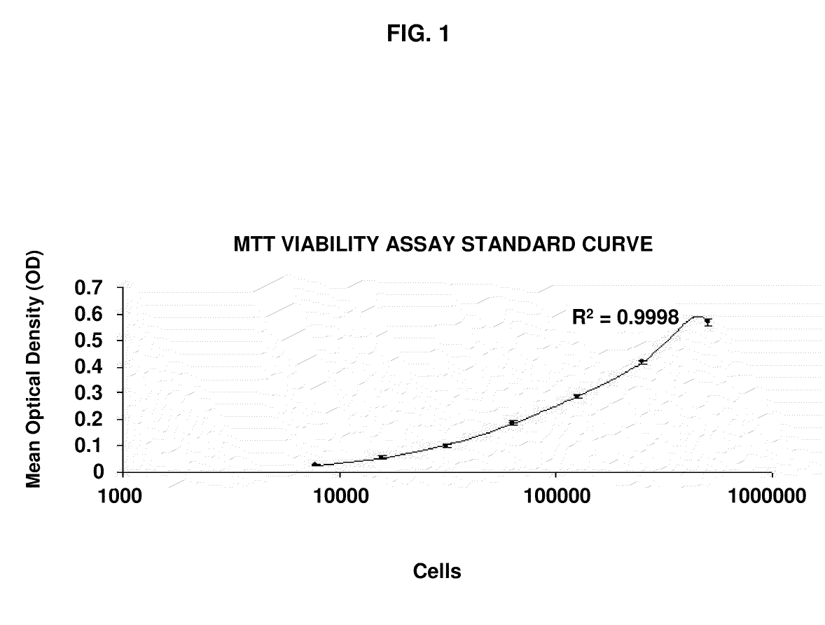

FIG. 1 illustrates the standard curve for MTT viability assay based on a 7-fold serial dilution with common factor 2. Maximum value is 500,000 cells. R² value represents 4-parametric regression. Values are represented as mean (n=3)±STD.

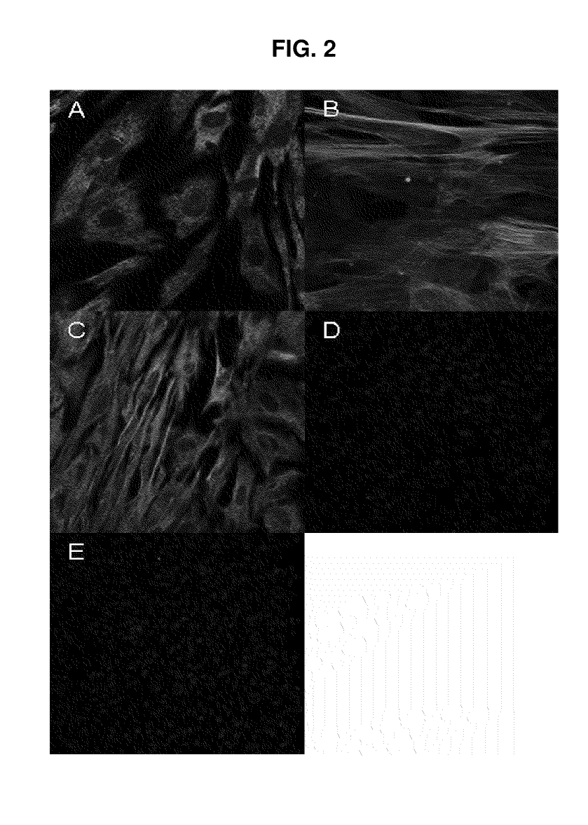

FIG. 2 illustrates human aortic valve leaflet interstitial cells showing myofibroblast phenotype. Immunofluorescent stain visualized with AlexaFluor 488. A. Vimentin positive (100×); B. alpha Smooth Muscle Actin positive (100×); C. Heat Shock Protein 47 positive (100×); D. Endothelial Nitric Oxide Synthase negative (20×); E. Negative control (Secondary antibody only, 20×) demonstrates the absence of non-specific fluorescence.

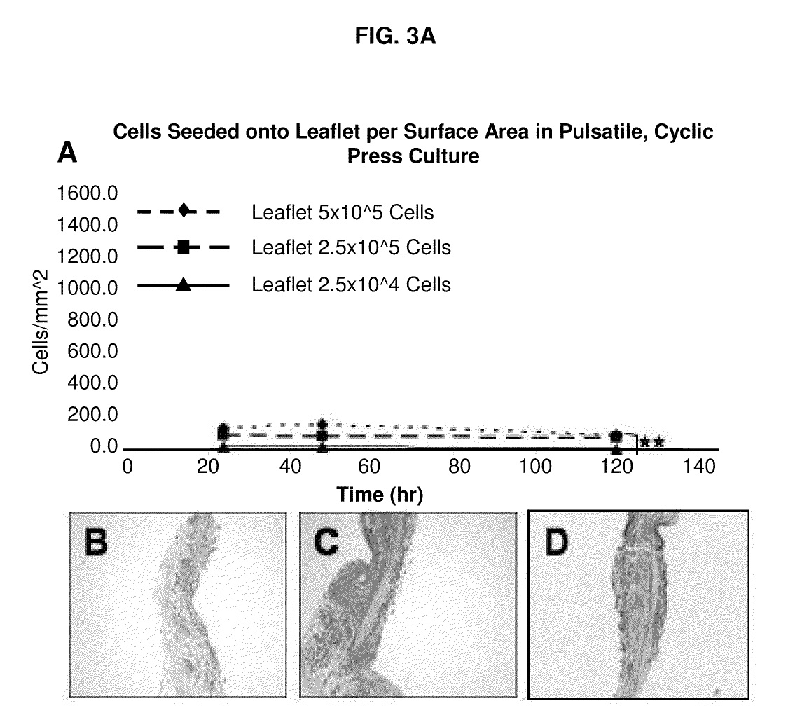

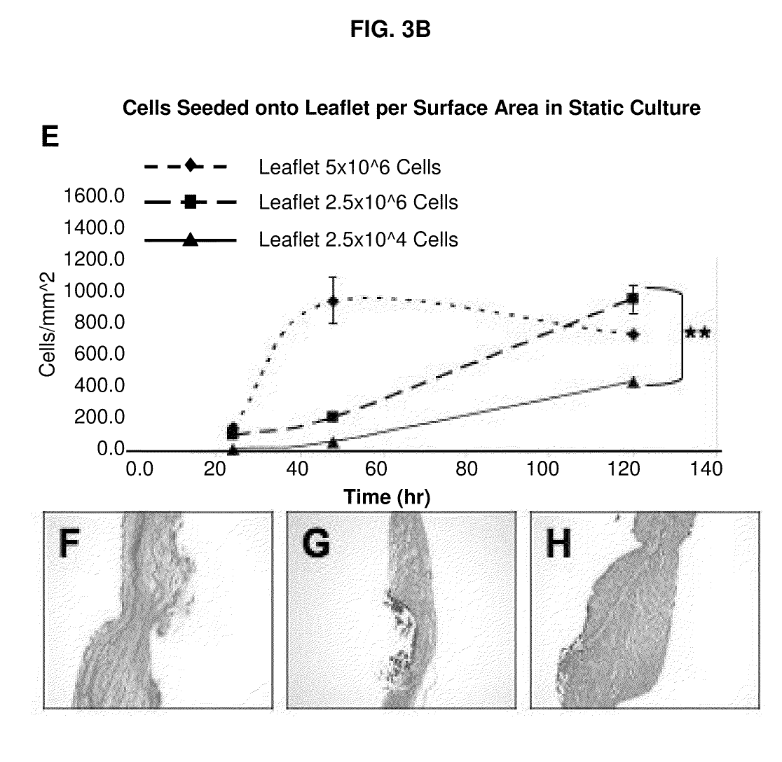

FIGS. 3A and 3B illustrate the number of viable, seeded cells on leaflet tissue at three seeding densities in (A) pulsatile culture and (E) static culture. Histological analysis shows increasing cell penetration from (B) 24 to (C) 48 to (D) 120 hours in pulsatile culture and increasing cell number on tissue surface from (F) 24 to (G) 48 to (H) 120 hours in static culture. Histology stained with H+E and imaged at 20×. Error reported as SEM. ** indicates statistical significance (p<0.05).

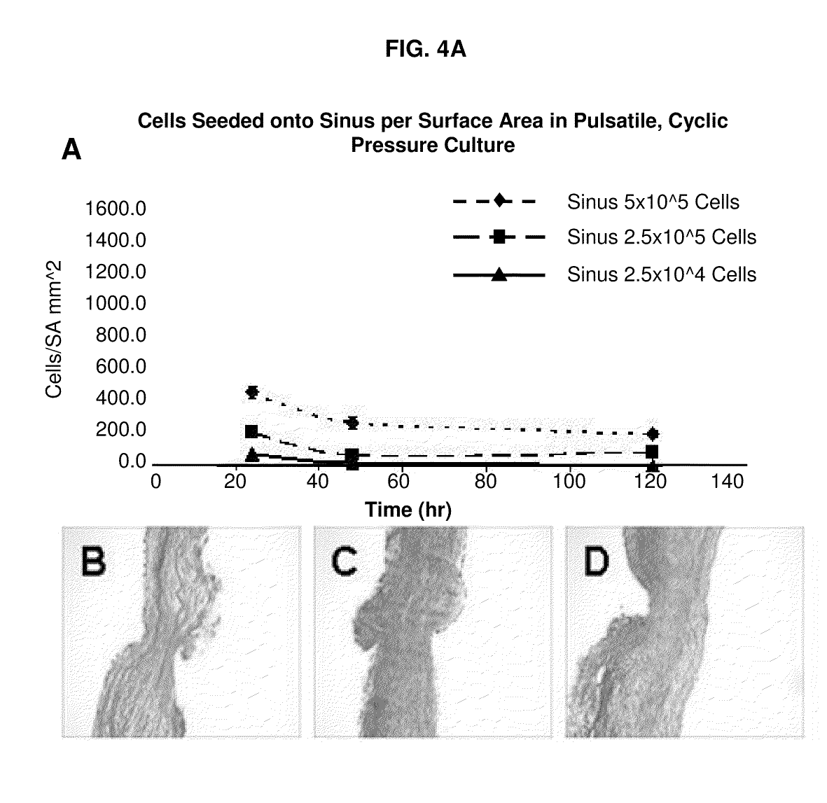

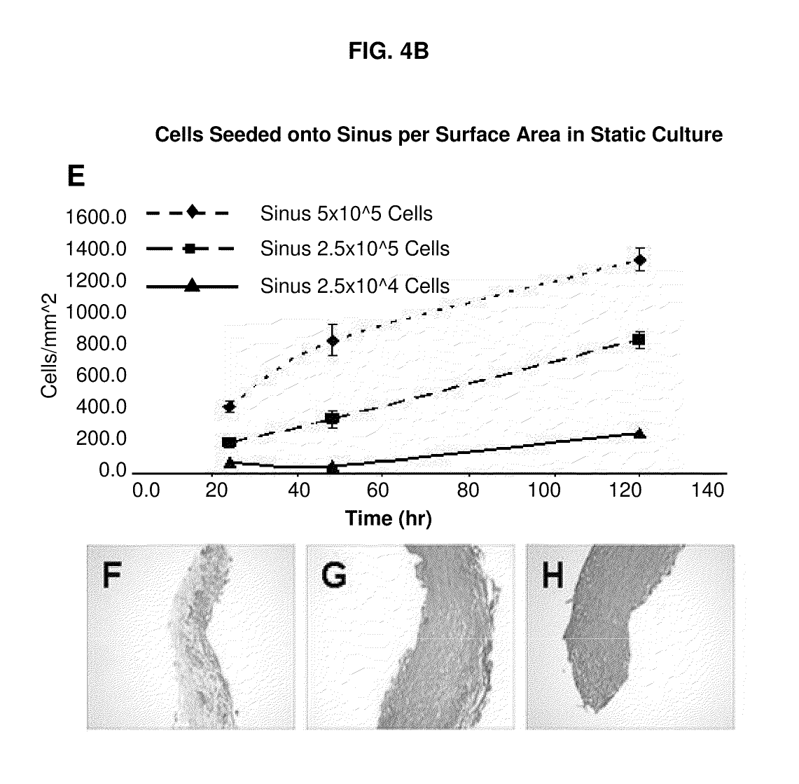

FIGS. 4A and 4B illustrate the number of viable, seeded cells on sinus tissue at three seeding densities in (A) pulsatile culture and (E) static culture. Histological analysis shows increasing cell penetration from (B) 24 to (C) 48 to (D) 120 hours in pulsatile culture and increasing cell number on tissue surface from (F) 24 to (G) 48 to (H) 120 hours in static culture. Histology stained with H+E and imaged at 20×. Error reported as SEM. * indicates statistical significance (p<0.05).

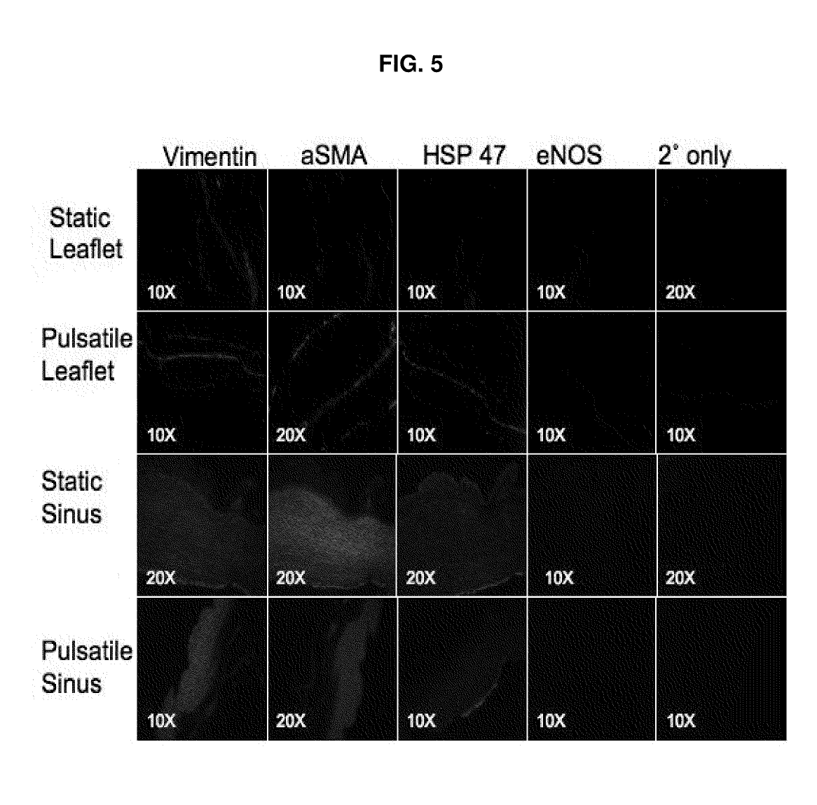

FIG. 5 illustrates immunohistochemical staining of leaflet and sinus tissue seeded for 5 days at highest dose. Positive staining for VIM, aSMA and HSP 47 in both pulsatile tissue types indicates active myofibroblasts. Equivalent negative stains in static tissue suggest quiescent fibroblasts. Negative eNOS stain precludes endothelial cell contamination. 2° antibody only is an internal negative control.

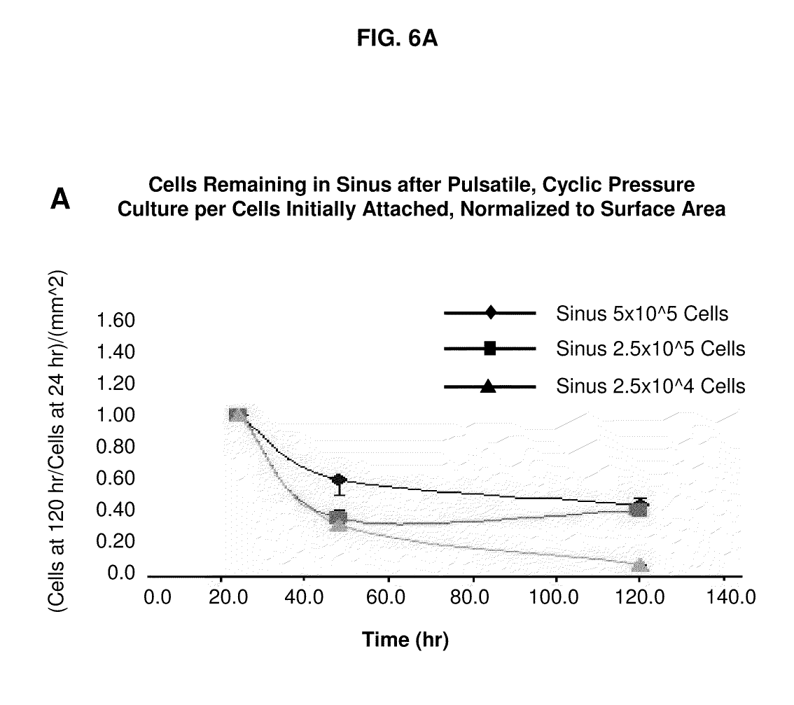

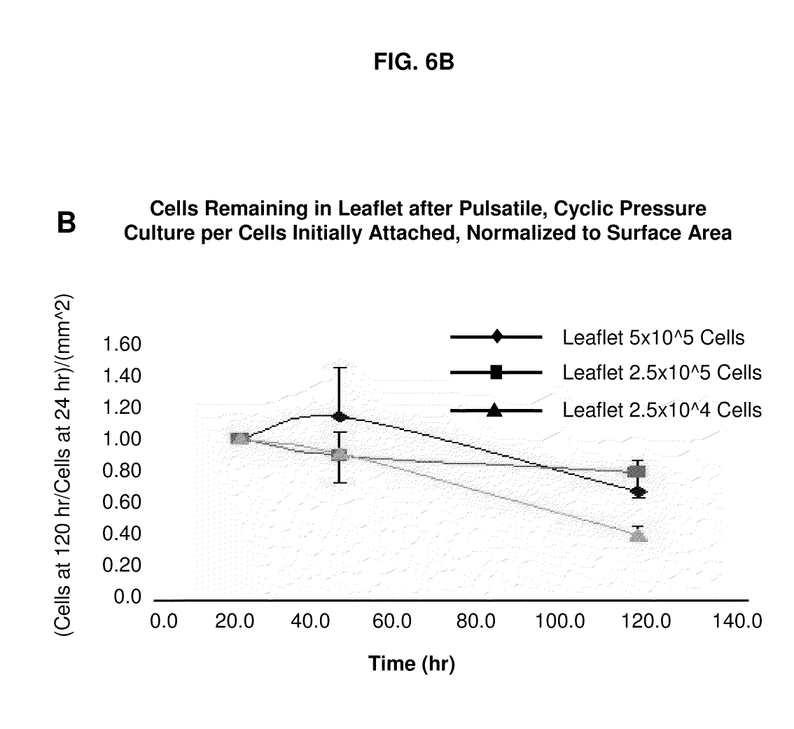

FIGS. 6A and 6B illustrate the number of viable, seeded cells on (A) sinus and (B) leaflet tissue after pulsatile, cyclic pressure culture per number of attached cells after 24 hours at three seeding densities, each normalized to surface area. Error reported as SEM. * indicates statistical significance (p<0.05) at 120 hour time point.

After five days in culture, leaflet tissues seeded with 2.5×10⁵ cells (median dose) and incubated in pulsatile culture were found to have an 11.2-fold decrease in cell number from the same time point in equivalent static assay. However, this quantitative decrease coincided with significant upregulation of cell motility and migration into the scaffold (FIGS. 3A and 3B).

At the same cell dose in sinus wall, a similar trend appeared; 120 hour pulsatile culture yielded a 10.6-fold decrease in cell number compared to static incubation (FIGS. 4A and 4B). Although the absolute quantitative difference varied, static culture always yielded significantly more total cells than pulsatile at each timepoint, for all cell doses, in both leaflet and sinus wall. Evaluation of the cell quantitative data in context with the histology suggests that this was primarily a consequence of significant surface cell proliferation (FIGS. 3A and 3B, FIGS. 4A and 4B) in the static environment.

Immunofluorescent labeling of cells growing in culture flasks revealed hVICs expressing Vimentin⁺, HSP 47⁺, a-SMA⁺, and eNOS⁻ (See FIG. 2). Immunohistochemistry of scaffolds incubated under static conditions for 5 days indicated phenotype expression of Vimentin⁺, HSP 47⁻, a-SMA⁻ and eNOS⁻, consistent with quiescent fibroblasts. Conversely, experimental scaffolds incubated under pulsatile conditions were found to be Vimentin⁺, HSP 47⁺, a-SMA⁺, and eNOS⁻, consistent with active myofibroblasts (See FIG. 5).

Vimentin⁺ and eNOS⁻ expression were seen across all tissue types, seeding doses and time points. At 24 and 48 hours of static seeding, HSP 47 and a-SMA showed faint positive staining that disappeared by 120 hours (data not shown). In pulsatile culture, HSP 47 and a-SMA staining appeared more intense progressing from 24 to 48 to 120 hours post seeding.

### Discussion and Conclusions

For leaflet and sinus wall, cyclic pressure incubation yielded fewer total cells associated with the scaffold than static incubation. However, the cells that remained after pulsatile culture had migrated into the interstitium and demonstrated enhanced matrix remodeling. Given that cells adherent to the surface detach from the scaffold shortly after implantation in vivo, protecting the cells from this fate by optimizing cyclic pressure-induced in vitro migration is an important variable affecting ultimate cell repopulation following orthotopic valve implantation.

In addition to its direct applications to seeding a TEHV, this assay has proven the feasibility of variably optimizing seeding conditions using valve biopsies; it can be used to individually test each facet of cell seeding. This methodology allowed for evaluating 576 individual scaffold biopsies under a controlled set of conditions with a single assay.

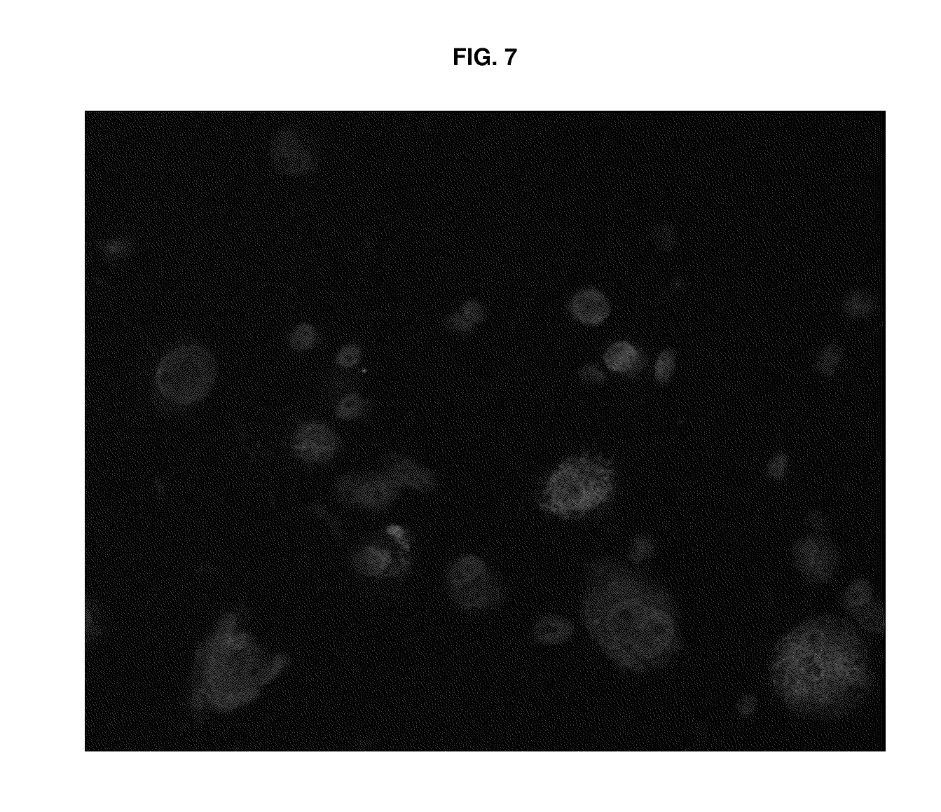

FIG. 7 shows cell numbers for sinus and leaflet in pulsatile culture normalized to 24 hour values, essentially removing the effect of initial seeding and relating the relative long-term advantages of adding additional cells at day 0. Given the absence of exponential divergence of the high dose from the middle dose, we concluded that 2.5×10⁵ cells should be sufficient to seed a 5×5 mm biopsy of valve tissue and that future optimization should focus on external conditions rather than simply higher initial cell seeding dose. In FIG. 7, the CD-68 positive staining (red) confirms macrophage differentiation following PMA 400×.

### Example 2

Currently approved clinical biological/bioprosthetic heart valve replacement options (allografts and xenografts) often result in reduced durability (likely due to innate inflammation and immune rejection and consequential calcification), ultimately leading to accelerated failure. Cryopreserved “viable” (i.e., containing donor cells) homografts as currently used are known to have limited durability due to inflammation and immune rejection resulting in fibrosis and calcification of the implanted valves resulting in valvular stenosis and/or insufficiency. Efficient decellularization can remove antigenic components from donor homograft valves, perhaps providing an antigen devoid of collagen/elastin extracellular matrix (ECM) scaffold that retains optimal structural elements of normal semilunar valves. Our group has previously demonstrated an absence of MHC-1 and MHC-2 positive antigenic debris following adequate decellularization of valves.

Decellularized homografts are clinically attractive as they surgically can be tailored homologously for size and location. They achieve immediate normal function post-implantation, and if not proinflammatory, may have the potential for prolonged durability. If such decellularized ECM valve scaffolds are not provocative of Inflammation other than of the non-immune wound healing type, then these may be suitable substrates for tissue engineering of viable valves (TEHVs) using ex vivo cell seeding and/or in vivo recellularization methods.

If decellularized heart valves do not induce significant inflammation, then they may be ideally suited for use as an ECM based tissue engineered heart valve scaffold.

### Materials and Methods

Inflammatory responses to putative scaffolds materials were compared at 0, 6, 24, 48 hours of challenge by measuring with ELISA six key cytokine titers: TNF-a; TGF-b1; IL-6; IL-2; IL-1b-1. Cytokine expression profiles were standardized as very low, low, moderate or high and compared using ANOVA (P £ 0.05=significant) statistical methods.

### Scaffolds Studied:

1. Cryopreserved (−180° Leaflets and sinus wall biopsies after the following treatments: DMSO clinical protocols) ovine aortic valves prepared with methods analogous to current clinical “viable” homograft valves designated: Fresh
2. Ovine aortic valves decellularized with a multisolvent, multidetergent, enzyme assisted, reciprocating osmolarity decellularization method: Decell
3. “Freeze Fractured” ovine aortic valves that were subjected to three rapid thaw (warm bath 37° C.) alternating with refreeze (without cryoprotectants) at −80° C. to freeze fracture the cells, thus maximizing antigen exposure (intracellular+cell surface sites): Freeze Fractured (FrFx)
4. Manufactured glutaraldehyde cross-linked porcine bioprosthetic valve leaflets obtained from Hancock® II clinical grade bioprosthetic valves: Hancock

### Results and Conclusions

Inflammatory responses as measured by human macrophage cytokine release profiles for decellularized ovine valve tissues were similar to glutaraldehyde fixed porcine leaflets despite the former being both xenogeneic and neither fixed nor cross-linked. These data suggest similar initial reductions in inflammatory potential can be achieved simply by removing all cells and cellular debris. In contrast to decellularized leaflets, both fresh- and freeze-fractured leaflets provoked elevations of TNF-a and IL-6 which are both important to initiating and regulating the innate immune response. In contrast to decellularized leaflets, both fresh- and freeze-fractured leaflets provoked elevations of TNF-a and IL-6 which are both important to initiating and regulating the innate immune response.

This assay provides an in vitro verification methodology for evaluating potential proinflammatory characteristics of materials and tissue-derived substrates being considered for use as TEHV scaffolds. Optimal scaffold candidates can then be more efficiently selected for evaluation with subsequent in vivo animal models.

Our study findings have demonstrated that the macrophage cytokine response to decellularized valve scaffolds is significantly reduced in comparison to the response induced by cryopreserved valves. The use of a decellularized valve, with reduced inflammatory potential, as an ECM scaffold for a tissue engineered heart valve is expected to result in a reduced incidence of fibrocalcification in the next generation of tissue replacement heart valves.

### Discussion

The muted inflammatory response evoked by porcine glutaraldehyde cross-linked prosthetic valve leaflets suggests that the satisfactory clinical experience in adults with these bioprostheses may be in part due to delayed inflammatory response. However, as the implant duration increases, a decrease in glutaraldehyde cross-linking density and an increased inflammatory response resulting in leaflet calcification and structural deterioration may occur.

Non-inflammatory mechanisms (e.g., elevated leaflet residual stresses, collagen bundle fracture) also contribute to the progressive loss of durability of cross-linked porcine bioprosthetic heart valves. Many of the currently implanted bioprosthetic valves have been designed to reduce the residual tissue stresses resulting in a reduction in structural deterioration.

Older theories as to the mechanisms for the limited durability of cross-linked xenograft bioprosthetic heart valves suggested failure modes focused on mechanisms related to physical and chemical deterioration leading to calcium accumulation and failure. More recent evidence suggests an important role for inflammation and immune mechanisms modulated by recipient factors such as age, immune competency, treatment with antirejection medications, etc., as the primary pathway to fibrocalcific degeneration. These data are consistent with a masking and unmasking of antigen sites.

These assay results suggest that decellularized valve scaffolds may have significantly reduced inflammatory potential and thus promise utility as platforms for tissue engineering replacement heart valves (TEHV). Since a putative clinical TEHV could be based on human ECM scaffolds rather than xenogeneic (e.g., ovine or porcine) results may have even been better than demonstrated in these experiments. Late protection might be conferred by the seeding of autologous valve interstitial cells capable of ECM protein degradation/synthesis and thus active constructive and perhaps adaptive remodeling.

### Example 3

Addressing mechanisms fundamentally related to native and prosthetic valve degeneration, the purpose of this study was to compare human macrophage cytokine provocation profiles of candidate materials for therapeutic use in reconstructing the human cardiovascular system and specifically for optimizing the scaffold component of tissue engineered cardiac valves as compared to current generation bioprosthetics for which clinical outcomes are well known. Because one attractive pathway for the development of a tissue engineered heart valve is the use of decellularized ECM valve scaffolds derived from cryopreserved cadaveric tissues, allograft and xenograft sources of valves were especially examined.

### Materials and Methods

### Test Materials Preparation

Human activated macrophage cytokine inflammatory responses over 48 hours were measured for biological samples obtained from porcine, ovine, and human aortic valves (nine valves for each species). Because of the significant differences in microstructure, leaflets and sinus wall samples were analyzed separately (e.g., absence of vascularity in leaflets, versus blood vessels, smooth muscle cells, pericytes and fibrocytes in vessel walls). The mammalian valve tissues were prepared in three ways with three valves allocated to each protocol. First, following aseptic harvest from juvenile animals, the ovine and porcine fresh valves were cryopreserved with preservation of cell viability utilizing a 10% DMSO in RPMI 1640 (Invitrogen, Carlsbad, Calif.) with 10% FBS (Invitrogen), cryopreservation at 1° C./min, a technique analogous to the preparation of current generation clinical cryopreserved heart valves (cell viability retained). These were designated as native, valve tissues further identified by species of origin. Another set of valves were harvested then subjected to freeze-thaw for three cycles of 21° C. to −80° C. without cryoprotectants. Cycled freeze-fracturing results in massive cell lysis, thereby potentially increasing overall antigen exposure. These were designated as freeze-fractured valve tissues. And finally, freshly cryopreserved animal heart valves were subjected to decellularization using a multi-solvent, multi-detergent, enzyme-assisted, reciprocating osmolarity method in which essentially all cells, cell debris and nuclear material were removed leaving only a structural protein ECM scaffold. These test materials were compared to two routinely used “inert” materials, nitinol and PTFE for which there is extensive clinical cardiovascular implant experience. Biopsies were obtained from each test valve from each of six sites (the three leaflets and the three sinus walls) and samples weighed for subsequent normalization of measurements to wet weight. Control samples were sized to equivalent weight samples as the biologics. Nitinol (sterile) was obtained from an Amplatzer™ size 5 mm Septal Occluder (SN 151438 A6A Medical Corporation, Plymouth, Minn., USA) and the PTFE was harvested from a sterile GorTex™ 4 mm thin wall vascular graft (W.L. Gore & Associates, Neward, Del., USA). Comparisons were made to materials from two current clinically used porcine glutaraldehyde crosslinked FDA-approved aortic valve bioprostheses (leaflets and sinus wall from aortic Freestyle® stentless and leaflets from the stented Hancock II®, both from Medtronic Corp., Minneapolis, Minn.). Human aortic valves were obtained at multi-organ and tissue harvests with informed consent for both research and clinical use, processed with cryopreservation under AATB guidelines by LifeNet Health Tissue Services (Virginia Beach, Va.) and stored at −180° C.; once out of date for clinical use, the valves were released for research use. These were prepared similarly to the test animal valves. All experimental animal materials were obtained with IACUC approval and in accordance with NIH, AALAS, and American Heart Association guidelines for the Care and Use of Research Animals.

### Macrophage Cytokine Assay

Human THP-1 monocytes (ATCC®-TIB-202™, Manassas, Va.), were obtained and prepared in suspension culture per ATCC protocol. Cell counts for each suspension were obtained using a automated cell counter (Coulter Counter®, Model Z3, Beckman Coulter, Inc., Fullerton, Calif.) and plated at a concentration of 1×10⁵ in each well of 24-well plates (Becton, Dickinson, Franklin Lakes, N.J.). Monocytes were differentiated into macrophages utilizing PMA/TPA (Phorbol 12-myristate 13 acetate: Sigma P8139, St. Louis, Mo.). Plates were incubated for 24 hours at 37° C., 21% O₂, 5% CO₂. Cell differentiation was confirmed with CD68 (Abcam-AB955, Cambridge, Mass.) immunocytochemical staining of a representative sample well from each plate and photographed (FIG. 7). Macrophages were incubated in RPMI 1640 with 10% FBS (THP1-growth media, Invitrogen™, Carlsbad, Calif.). Two hours prior to assay, the macrophages were activated with LPS (lipopolysaccharides-Sigma L6261, St. Louis, Mo.). Baseline cytokine titers (time 0) were obtained to establish the priming effect and were subtracted from the subsequent measurements to calculate the provoked cytokine expression. Each prepared test specimen was placed in its respective well of 24 well plates. At the defined timepoints (6, 24, 48 hours) the entire supernatant from wells were harvested and each well used for a single ELISA. Each data point was determined using three wells/cytokine for 6 biopsies of the test type (ie, n=18 determinations). Control and test biomaterials were assayed for all five cytokines at each timepoint. Cell supernatants were obtained and frozen at −80° C. After collection of all supernatant samples, the cytokine assays were analyzed in batches by ELISA in triplicate for TNF-α, IL-2, IL-6, TGF-β1 and IL-1-β1 (Quantikine® Assay kits, R&D Systems®, Minneapolis, Minn.). Curves were constructed from measurements using standard controls. Dilution expression standards were measured with each assay run per kit instructions. Quantification was performed at wavelengths of 570 nm and 450 nm (correction wavelength) after twenty minutes incubation with the specific cytokine conjugate substrate. Based on reference standards provided by manufacturer, each cytokine titer was then ranked on its own standardized expression scale from very low to very high.

Nine aortic valves from each of three mammalian species (n=27) were randomized in groups of three to each of the three preparation methods. Leaflet and sinus wall tissues were separately analyzed by assaying six separate tissue samples randomly biopsied from all 3 leaflets and sinuses. Titers were measured at three timepoints (6, 24, 48 hours after zero baseline). Each of the five cytokine assays was run in triplicate. Total assay determinations n=6480. Six samples from each of the “inert” controls (PTFE, nitinol) and the two bioprosthetic valve materials were assayed at the three timepoints with each cytokine specific ELISA performed in triplicate (n=1800).

### Histology

Standard sections of each of the test materials were prepared with HistoChoice™ MB Fixative (Amresco, Inc., Solon, Ohio), paraffin embedded and examined histologically with Hematoxylin-Eosin (H&E) staining. Collagen and elastin structures were visualized with Movat's pentachrome staining (FIG. 6). (Mastertechs, Lodi/Calif., USA)

### DNA Content

The decellularization process was evaluated in an additional six ovine valves by measuring residual DNA as compared to native fresh aortic valves using a dsDNA High-Sensitivity assay kit (Quant-it™, Invitrogen, Carlsbad, Calif.), with each measurement in triplicate. The histology and DNA quantification verified essentially complete decellularization (Table 1).

### Statistics

Continuous variables were analyzed for differences between groups with two-way ANOVA (Tukey Kramer and Kruskal-Wallis tests) while ordinal variable were analyzed with Wilcox Rank Sum. Student's T-test was used for paired single comparisons (eg, DNA content before and after decellularization). (SPSS v.17) P≦0.05 was considered significant. All cytokine quantifications are reported in the tables, but because wall and leaflets tracked similarly, for clarity the reported figures display leaflet comparisons and statistical tests unless otherwise noted.

### Results and Conclusions

TABLE 2
DNA Quantification Assessing Decellularization Method in
Ovine Valves

| | | | Aortic |
|---|---|---|---|
| | Leaflets | Sinus Wall | Wall above valve |
| Native valves | 0.794 ± 0.176* | 0.557 ± 0.074* | 0.586 ± 0.119* |
| Decellularized | 0.000 ± 0.000 | 0.000 ± 0.000 | 0.001 ± 0.001 |
| valves | | | |

*DNA ugm/mg tissue weight ± 1 S.D. Students T-test native material versus decell
*Note that 0 reflects values below the detection limits of the assay.

### Cytokine Provocation for Each Biomaterial

The ovine and porcine tissues provoked higher cytokine protein expression than did human tissues (Table 3). The highest titers tended to be at six hours with decay over the ensuing 48 hours. Decellularization reduced the provocation for all mammalian types but especially so for human tissues. The “inert” materials and glutaraldehyde-treated porcine valve prosthetics had very low titers which were closely matched by the decellularized human tissues. For each tissue type, and processing method at each timepoint, the leaflet and sinus wall results trended similarly but the titers for wall samples tended to be slightly higher than their analogous leaflets. Elevations of TGFβ1, TNFα and IL2 had the more prolonged expression profiles whereas IL1β1 and IL6 typically had returned close to baseline by 48 hours. Freeze-fracturing of the biological materials consistently generated the highest cytokine levels. Decellularized human tissues provoked much lower cytokine signaling than did the porcine or ovine decellularized materials.

TABLE 3
Cytokine protein expression for mammalian native, freeze fractured, and decellularized ECM
scaffolds as compared to “inert” and glutaraldehyde crosslinked bioprosthetic materials

| Cytokine | | | | | | | | |
|---|---|---|---|---|---|---|---|---|
| TGF β-1 | | | TNF-α | | | IL-6 | | |
| Timepoint | | | | | | | | |
| 6 H | 24 H | 48 H | 6 H | 24 H | 48 H | 6 H | 24 H | 48 H |

HUMAN AORTIC VALVE

| | | | | | | | | | |
|---|---|---|---|---|---|---|---|---|---|
| Decellularized Leaflet | 86.6 ± 3.5 | 59.7 ± 3.1 | 51.4 ± 2.3 | 45.3 ± 2.8 | 10.3 ± 1.3 | 10.1 ± 1.2 | 31.7 ± 4.3 | 20.0 ± 2.9 | 9.3 ± 1.7 |
| Decellularized Sinus | 92.8 ± 4.0 | 63.3 ± 3.1 | 50.8 ± 3.4 | 43.1 ± 3.5 | 19.4 ± 3.9 | 18.6 ± 1.7 | 30.7 ± 3.3 | 28.2 ± 3.6 | 10.2 ± 2.0 |
| Wall | | | | | | | | | |
| Native Leaflet | 600.3 ± 15.9 | 257.4 ± 9.3 | 190.8 ± 10.7 | 124.0 ± 9.6 | 82.7 ± 5.7 | 57.0 ± 5.2 | 91.9 ± 3.8 | 68.6 ± 8.1 | 39.7 ± 3.9 |
| Native Sinus Wall | 609.8 ± 14.8 | 272.7 ± 8.3 | 201.7 ± 13.0 | 136.6 ± 6.4 | 92.1 ± 6.2 | 58.1 ± 6.0 | 99.3 ± 6.1 | 69.1 ± 4.9 | 43.4 ± 3.2 |
| Freeze Fractured | 862.5 ± 13.9 | 660.8 ± 25.3 | 505.7 ± 22.2 | 207.0 ± 21.7 | 142.8 ± 12.8 | 93.8 ± 11.6 | 119.8 ± 11.0 | 68.6 ± 5.2 | 52.7 ± 5.5 |
| Leaflet | | | | | | | | | |
| Freeze Fractured Sinus | 867.1 ± 19.5 | 686.8 ± 26.0 | 522.9 ± 21.9 | 243.3 ± 17.5 | 182.4 ± 17.0 | 114.0 ± 11.9 | 126.7 ± 7.1 | 71.2 ± 7.3 | 42.2 ± 3.3 |
| Wall | | | | | | | | | |

PORCINE AORTIC VALVE

| | | | | | | | | | |
|---|---|---|---|---|---|---|---|---|---|
| Decellularized Leaflet | 211.4 ± 13.1 | 161.2 ± 23.5 | 121.7 ± 9.4 | 99.7 ± 3.7 | 85.6 ± 8.0 | 68.6 ± 4.8 | 86.5 ± 6.8 | 57.5 ± 4.9 | 14.8 ± 1.3 |
| Decellularized Sinus | 218.6 ± 11.9 | 182.3 ± 9.7 | 138.2 ± 12.8 | 100.1 ± 4.0 | 87.8 ± 9.6 | 71.0 ± 5.0 | 92.3 ± 6.4 | 70.0 ± 3.9 | 24.2 ± 2.8 |
| Wall | | | | | | | | | |
| Native Leaflet | 719.7 ± 14.6 | 437.0 ± 10.7 | 284.8 ± 9.6 | 175.5 ± 7.4 | 151.7 ± 10.6 | 74.8 ± 7.5 | 108.4 ± 8.8 | 90.7 ± 4.4 | 50.1 ± 4.5 |
| Native Sinus Wall | 786.4 ± 9.6 | 493.6 ± 15.0 | 268.6 ± 14.7 | 179.4 ± 8.3 | 165.5 ± 13.2 | 90.9 ± 5.4 | 112.8 ± 8.5 | 99.6 ± 6.1 | 59.5 ± 5.0 |
| Freeze Fractured | 903.2 ± 26.0 | 720.9 ± 31.6 | 597.4 ± 27.5 | 293.4 ± 20.8 | 278.3 ± 20.5 | 211.8 ± 13.6 | 139.4 ± 15.7 | 90.2 ± 13.1 | 66.5 ± 3.3 |
| Leaflet | | | | | | | | | |
| Freeze Fractured Sinus | 912.2 ± 30.3 | 753.5 ± 26.5 | 607.4 ± 26.4 | 306.2 ± 24.0 | 295.2 ± 37.3 | 255.3 ± 11.8 | 151.1 ± 14.5 | 101.2 ± 8.4 | 68.7 ± 5.2 |
| Wall | | | | | | | | | |

OVINE AORTIC VALVE

| | | | | | | | | | |
|---|---|---|---|---|---|---|---|---|---|
| Decellularized Leaflet | 182.4 ± 5.6 | 189.9 ± 4.4 | 224.0 ± 7.2 | 59.1 ± 5.5 | 60.9 ± 4.6 | 62.3 ± 4.2 | 89.4 ± 5.0 | 58.9 ± 4.5 | 0.0 ± 0.0 |
| Decellularized Sinus | 206.2 ± 5.5 | 221.4 ± 9.2 | 284.8 ± 6.9 | 97.1 ± 4.7 | 110.7 ± 5.5 | 108.5 ± 5.5 | 100.6 ± 6.0 | 64.0 ± 5.8 | 0.0 ± 0.0 |
| Wall | | | | | | | | | |
| Native Leaflet | 255.5 ± 10.4 | 240.6 ± 12.3 | 149.2 ± 5.9 | 101.8 ± 7.2 | 108.2 ± 8.1 | 89.6 ± 4.3 | 73.4 ± 5.0 | 20.6 ± 3.1 | 0.0 ± 0.0 |
| Native Sinus Wall | 281.7 ± 5.2 | 243.7 ± 6.1 | 168.2 ± 6.7 | 124.4 ± 8.2 | 134.3 ± 7.3 | 99.7 ± 5.2 | 89.0 ± 6.0 | 40.1 ± 5.1 | 0.0 ± 0.0 |
| Freeze Fractured | 647.5 ± 7.6 | 511.1 ± 12.3 | 231.3 ± 8.8 | 188.0 ± 9.2 | 233.1 ± 10.4 | 184.9 ± 10.3 | 123.2 ± 7.0 | 49.9 ± 5.6 | 23.7 ± 3.6 |
| Leaflet | | | | | | | | | |
| Freeze Fractured Sinus | 844.1 ± 17.7 | 604.2 ± 14.5 | 288.1 ± 10.0 | 203.9 ± 10.1 | 247.8 ± 14.7 | 154.8 ± 12.2 | 153.8 ± 7.9 | 72.4 ± 5.0 | 22.0 ± 4.6 |
| Wall | | | | | | | | | |

INERT MATERIALS

| | | | | | | | | | |
|---|---|---|---|---|---|---|---|---|---|
| PTFE | 49.8 ± 2.0 | 0.0 ± 0.0 | 0.0 ± 0.0 | 9.9 ± 0.6 | 0.0 ± 0.0 | 0.0 ± 0.0 | 15.8 ± 0.9 | 5.0 ± 0.8 | 0.0 ± 0.0 |
| Nitinol | 38.2 ± 1.6 | 0.0 ± 0.0 | 0.0 ± 0.0 | 13.8 ± 0.8 | 0.0 ± 0.0 | 0.0 ± 0.0 | 16.9 ± 1.0 | 3.1 ± 0.6 | 0.0 ± 0.0 |

PORCINE BIOPROSTHETICS
Glutaraldehyde treated

| | | | | | | | | | |
|---|---|---|---|---|---|---|---|---|---|
| Stentless Leaflet | 46.1 ± 6.4 | 40.2 ± 2.4 | 26.5 ± 2.5 | 9.7 ± 1.6 | 9.6 ± 1.6 | 6.3 ± 1.6 | 12.4 ± 2.0 | 8.2 ± 1.2 | 0.0 ± 0.0 |
| Stentless Sinus | 53.7 ± 8.1 | 48.4 ± 4.2 | 30.3 ± 3.7 | 14.7 ± 2.0 | 11.2 ± 1.7 | 7.7 ± 1.4 | 22.6 ± 2.9 | 12.4 ± 1.9 | 0.0 ± 0.0 |
| Stented Leaflet | 88.0 ± 3.9 | 80.4 ± 3.1 | 73.9 ± 4.4 | 12.3 ± 2.5 | 10.6 ± 2.7 | 10.3 ± 2.3 | 19.3 ± 2.5 | 18.2 ± 1.7 | 0.0 ± 0.0 |

| | Cytokine | | | | | |
|---|---|---|---|---|---|---|
| | IL-2 | | | IL-1 β1 | | |
| | Timepoint | | | | | |
| | 6 H | 24 H | 48 H | 6 H | 24 H | 48 H |
| HUMAN AORTIC VALVE | | | | | | |
| Decellularized Leaflet | 154.4 ± 4.9 | 124.1 ± 9.3 | 89.3 ± 5.4 | 20.1 ± 2.9 | 0.0 ± 0.0 | 0.0 ± 0.0 |
| Decellularized Sinus Wall | 163.2 ± 4.4 | 137.0 ± 6.2 | 92.2 ± 4.6 | 19.8 ± 2.7 | 0.0 ± 0.0 | 0.0 ± 0.0 |
| Native Leaflet | 418.7 ± 9.0 | 194.6 ± 6.2 | 91.4 ± 4.4 | 90.8 ± 12.0 | 19.8 ± 2.5 | 0.0 ± 0.0 |
| Native Sinus Wall | 446.7 ± 12.8 | 223.3 ± 8.9 | 125.7 ± 9.5 | 94.3 ± 7.6 | 26.8 ± 4.3 | 0.0 ± 0.0 |
| Freeze Fractured Leaflet | 786.7 ± 11.0 | 624.3 ± 8.5 | 205.6 ± 12.0 | 99.8 ± 12.5 | 56.6 ± 9.8 | 0.0 ± 0.0 |
| Freeze Fractured Sinus Wall | 801.6 ± 15.4 | 641.7 ± 11.2 | 242.6 ± 12.6 | 102.1 ± 11.3 | 63.8 ± 11.8 | 0.0 ± 0.0 |
| PORCINE AORTIC VALVE | | | | | | |
| Decellularized Leaflet | 397.3 ± 10.2 | 300.7 ± 12.9 | 190.0 ± 5.3 | 41.6 ± 5.7 | 28.4 ± 3.3 | 0.0 ± 0.0 |
| Decellularized Sinus Wall | 506.1 ± 28.6 | 324.1 ± 9.2 | 210.5 ± 10.8 | 48.3 ± 5.2 | 30.4 ± 3.5 | 0.0 ± 0.0 |
| Native Leaflet | 625.7 ± 10.1 | 534.9 ± 8.2 | 107.2 ± 6.2 | 100.7 ± 11.9 | 64.5 ± 4.1 | 0.0 ± 0.0 |
| Native Sinus Wall | 646.0 ± 9.5 | 541.4 ± 10.1 | 121.1 ± 8.0 | 109.4 ± 6.2 | 71.0 ± 4.8 | 3.9 ± 1.1 |
| Freeze Fractured Leaflet | 913.6 ± 17.1 | 828.4 ± 13.9 | 212.4 ± 8.0 | 132.3 ± 11.4 | 85.2 ± 9.0 | 7.8 ± 1.8 |
| Freeze Fractured Sinus Wall | 902.3 ± 13.2 | 854.9 ± 14.3 | 222.6 ± 10.6 | 137.4 ± 14.8 | 93.4 ± 15.7 | 10.7 ± 1.7 |
| OVINE AORTIC VALVE | | | | | | |
| Decellularized Leaflet | 314.4 ± 8.2 | 286.8 ± 8.4 | 209.1 ± 12.3 | 46.6 ± 3.8 | 21.9 ± 3.8 | 0.0 ± 0.0 |
| Decellularized Sinus Wall | 553.5 ± 9.0 | 400.8 ± 7.8 | 205.7 ± 11.4 | 45.5 ± 5.5 | 19.4 ± 3.2 | 0.0 ± 0.0 |
| Native Leaflet | 204.1 ± 13.2 | 264.7 ± 6.4 | 101.9 ± 9.3 | 50.7 ± 6.4 | 8.6 ± 1.8 | 0.0 ± 0.0 |
| Native Sinus Wall | 275.8 ± 6.4 | 351.4 ± 7.3 | 122.9 ± 11.1 | 53.5 ± 5.5 | 7.2 ± 1.6 | 0.0 ± 0.0 |
| Freeze Fractured Leaflet | 941.1 ± 18.3 | 954.8 ± 15.0 | 201.3 ± 14.2 | 107.9 ± 8.1 | 56.9 ± 5.4 | 0.0 ± 0.0 |
| Freeze Fractured Sinus Wall | 1203.4 ± 22.2 | 1159.9 ± 12.4 | 288.8 ± 22.0 | 125.6 ± 12.0 | 51.0 ± 6.8 | 0.0 ± 0.0 |
| INERT MATERIALS | | | | | | |
| PTFE | 89.2 ± 2.4 | 9.9 ± 0.8 | 0.0 ± 0.0 | 6.8 ± 0.9 | 0.0 ± 0.0 | 0.0 ± 0.0 |
| Nitinol | 90.6 ± 2.0 | 19.3 ± 1.9 | 0.0 ± 0.0 | 8.0 ± 0.7 | 0.0 ± 0.0 | 0.0 ± 0.0 |
| PORCINE BIOPROSTHETICS | | | | | | |
| Glutaraldehyde treated | | | | | | |
| Stentless Leaflet | 116.5 ± 7.3 | 90.2 ± 3.1 | 71.7 ± 4.1 | 11.9 ± 2.0 | 0.0 ± 0.0 | 0.0 ± 0.0 |
| Stentless Sinus | 124.3 ± 10.3 | 99.5 ± 6.3 | 80.5 ± 4.2 | 13.4 ± 2.0 | 0.0 ± 0.0 | 0.0 ± 0.0 |
| Stented Leaflet | 173.4 ± 5.3 | 188.4 ± 4.8 | 123.1 ± 9.9 | 7.7 ± 1.6 | 0.0 ± 0.0 | 0.0 ± 0.0 |

TABLE 4
Cytokine protein expression for mammalian native, freeze fractured, and decellularized
ECM scaffolds as compared to “inert” and glutaraldehyde crosslinked bioprosthetic materials

| Cytokine | | | | | | | | |
|---|---|---|---|---|---|---|---|---|
| TGF β-1 | | | TNF-α | | | IL-6 | | |
| Timepoint | | | | | | | | |
| 6 H | 24 H | 48 H | 6 H | 24 H | 48 H | 6 H | 24 H | 48 H |

HUMAN AORTIC VALVE

| | | | | | | | | | |
|---|---|---|---|---|---|---|---|---|---|
| Decellularized Leaflet | 86.6 ± 3.5 | 59.7 ± 3.1 | 51.4 ± 2.3 | 45.3 ± 2.8 | 10.3 ± 1.3 | 10.1 ± 1.2 | 31.7 ± 4.3 | 20.0 ± 2.9 | 9.3 ± 1.7 |
| Decellularized Sinus | 92.8 ± 4.0 | 63.3 ± 3.1 | 50.8 ± 3.4 | 43.1 ± 3.5 | 19.4 ± 3.9 | 18.6 ± 1.7 | 30.7 ± 3.3 | 28.2 ± 3.6 | 10.2 ± 2.0 |
| Wall | | | | | | | | | |
| Native Leaflet | 600.3 ± 15.9 | 257.4 ± 9.3 | 190.8 ± 10.7 | 124.0 ± 9.6 | 82.7 ± 5.7 | 57.0 ± 5.2 | 91.9 ± 3.8 | 68.6 ± 8.1 | 39.7 ± 3.9 |
| Native Sinus Wall | 609.8 ± 14.8 | 272.7 ± 8.3 | 201.7 ± 13.0 | 136.6 ± 6.4 | 92.1 ± 6.2 | 58.1 ± 6.0 | 99.3 ± 6.1 | 69.1 ± 4.9 | 43.4 ± 3.2 |
| Freeze Fractured | 862.5 ± 13.9 | 660.8 ± 25.3 | 505.7 ± 22.2 | 207.0 ± 21.7 | 142.8 ± 12.8 | 93.8 ± 11.6 | 119.8 ± 11.0 | 68.6 ± 5.2 | 52.7 ± 5.5 |
| Leaflet | | | | | | | | | |
| Freeze Fractured Sinus | 867.1 ± 19.5 | 686.8 ± 26.0 | 522.9 ± 21.9 | 243.3 ± 17.5 | 182.4 ± 17.0 | 114.0 ± 11.9 | 126.7 ± 7.1 | 71.2 ± 7.3 | 42.2 ± 3.3 |
| Wall | | | | | | | | | |

PAPIO AORTIC VALVE

| | | | | | | | | | |
|---|---|---|---|---|---|---|---|---|---|
| Decellularized Leaflet | 93.8 ± 1.4 | 68.8 ± 3.3 | 60.4 ± 3.4 | 75.3 ± 2.7 | 25.8 ± 1.2 | 15.9 ± 1.1 | 41.9 ± 3.0 | 25.8 ± 2.5 | 10.0 ± 0.9 |
| Decellularized Sinus | 100.8 ± 1.6 | 75.9 ± 5.0 | 62.8 ± 4.0 | 77.8 ± 2.5 | 30.1 ± 2.0 | 18.7 ± 1.8 | 45.1 ± 3.4 | 27.2 ± 2.5 | 10.7 ± 1.3 |
| Wall | | | | | | | | | |
| Native Leaflet | 622.9 ± 5.9 | 301.6 ± 8.5 | 248.8 ± 7.1 | 134.3 ± 4.0 | 100.8 ± 5.3 | 59.6 ± 4.4 | 104.9 ± 4.7 | 78.8 ± 4.4 | 55.3 ± 3.8 |
| Native Sinus Wall | 622.3 ± 10.4 | 325.7 ± 7.1 | 254.4 ± 6.2 | 149.4 ± 5.3 | 115.1 ± 7.9 | 72.8 ± 4.2 | 117.2 ± 8.4 | 82.9 ± 4.4 | 60.9 ± 4.3 |
| Freeze Fractured | 898.3 ± 18.7 | 673.7 ± 19.7 | 578.9 ± 25.8 | 229.2 ± 5.9 | 166.2 ± 14.6 | 103.2 ± 11.0 | 133.1 ± 9.7 | 93.4 ± 8.8 | 64.5 ± 6.8 |
| Leaflet | | | | | | | | | |
| Freeze Fractured Sinus | 899.6 ± 22.1 | 694.2 ± 18.0 | 629.3 ± 23.5 | 253.4 ± 11.5 | 197.2 ± 14.5 | 121.0 ± 14.7 | 152.4 ± 17.4 | 112.6 ± 10.0 | 69.0 ± 6.4 |
| Wall | | | | | | | | | |

PORCINE BIOPROSTHETICS
Glutaraldehyde treated

| | | | | | | | | | |
|---|---|---|---|---|---|---|---|---|---|
| Stented Leaflet MAC2 | 88.0 ± 3.9 | 80.4 ± 3.1 | 73.9 ± 4.4 | 12.3 ± 2.5 | 10.6 ± 2.7 | 10.3 ± 2.3 | 19.3 ± 2.5 | 18.2 ± 1.7 | 0.0 ± 0.0 |
| Stentless Leaflet | 46.1 ± 6.4 | 40.2 ± 2.4 | 26.5 ± 2.5 | 9.7 ± 1.6 | 9.6 ± 1.6 | 6.3 ± 1.6 | 12.4 ± 2.0 | 8.2 ± 1.2 | 0.0 ± 0.0 |
| MAC2 | | | | | | | | | |
| Stentless Sinus MAC2 | 53.7 ± 8.1 | 48.4 ± 4.2 | 30.3 ± 3.7 | 14.7 ± 2.0 | 11.2 ± 1.7 | 7.7 ± 1.4 | 22.6 ± 2.9 | 12.4 ± 1.9 | 0.0 ± 0.0 |
| Stentless Leaflet | 58.7 ± 3.2 | 49.5 ± 4.5 | 31.0 ± 2.8 | 8.8 ± 0.7 | 7.1 ± 0.7 | 7.0 ± 0.6 | 18.2 ± 1.3 | 11.8 ± 1.4 | 0.0 ± 0.0 |
| MAC3 | | | | | | | | | |
| Stentless Sinus MAC3 | 68.7 ± 2.0 | 57.8 ± 5.1 | 29.2 ± 2.8 | 13.7 ± 1.2 | 8.9 ± 0.8 | 7.2 ± 0.6 | 23.6 ± 1.8 | 18.0 ± 1.3 | 0.0 ± 0.0 |

INERT MATERIALS

| | | | | | | | | | |
|---|---|---|---|---|---|---|---|---|---|
| PTFE | 49.8 ± 2.0 | 0.0 ± 0.0 | 0.0 ± 0.0 | 9.9 ± 0.6 | 0.0 ± 0.0 | 0.0 ± 0.0 | 15.8 ± 0.9 | 5.0 ± 0.8 | 0.0 ± 0.0 |
| Nitinol | 38.2 ± 1.6 | 0.0 ± 0.0 | 0.0 ± 0.0 | 13.8 ± 0.8 | 0.0 ± 0.0 | 0.0 ± 0.0 | 16.9 ± 1.0 | 3.1 ± 0.6 | 0.0 ± 0.0 |

| Cytokine | |
|---|---|
| IL-2 | IL-1 β1 |

| | Timepoint | | | | | |
|---|---|---|---|---|---|---|
| | 6 H | 24 H | 48 H | 6 H | 24 H | 48 H |
| HUMAN AORTIC VALVE | | | | | | |
| Decellularized Leaflet | 154.4 ± 4.9 | 124.1 ± 9.3 | 89.3 ± 5.4 | 20.1 ± 2.9 | 0.0 ± 0.0 | 0.0 ± 0.0 |
| Decellularized Sinus Wall | 163.2 ± 4.4 | 137.0 ± 6.2 | 92.2 ± 4.6 | 19.8 ± 2.7 | 0.0 ± 0.0 | 0.0 ± 0.0 |
| Native Leaflet | 418.7 ± 9.0 | 194.6 ± 6.2 | 91.4 ± 4.4 | 90.8 ± 12.0 | 19.8 ± 2.5 | 0.0 ± 0.0 |
| Native Sinus Wall | 446.7 ± 12.8 | 223.3 ± 8.9 | 125.7 ± 9.5 | 94.3 ± 7.6 | 26.8 ± 4.3 | 0.0 ± 0.0 |
| Freeze Fractured Leaflet | 786.7 ± 11.0 | 624.3 ± 8.5 | 205.6 ± 12.0 | 99.8 ± 12.5 | 56.6 ± 9.8 | 0.0 ± 0.0 |
| Freeze Fractured Sinus Wall | 801.6 ± 15.4 | 641.7 ± 11.2 | 242.6 ± 12.6 | 102.1 ± 11.3 | 63.8 ± 11.8 | 0.0 ± 0.0 |
| PAPIO AORTIC VALVE | | | | | | |
| Decellularized Leaflet | 164.2 ± 3.6 | 139.3 ± 4.5 | 99.8 ± 3.4 | 21.9 ± 1.1 | 0.0 ± 0.0 | 0.0 ± 0.0 |
| Decellularized Sinus Wall | 170.7 ± 4.8 | 145.8 ± 5.5 | 103.9 ± 4.8 | 23.9 ± 1.0 | 0.0 ± 0.0 | 0.0 ± 0.0 |
| Native Leaflet | 430.4 ± 13.7 | 257.3 ± 10.8 | 137.2 ± 8.4 | 94.4 ± 2.4 | 24.7 ± 3.4 | 0.0 ± 0.0 |
| Native Sinus Wall | 452.6 ± 14.9 | 241.4 ± 9.0 | 141.7 ± 8.0 | 95.5 ± 1.4 | 32.3 ± 2.8 | 0.0 ± 0.0 |
| Freeze Fractured Leaflet | 802.3 ± 16.4 | 651.8 ± 15.9 | 247.3 ± 18.1 | 104.6 ± 8.4 | 64.4 ± 6.1 | 0.0 ± 0.0 |
| Freeze Fractured Sinus Wall | 805.0 ± 16.9 | 658.0 ± 15.6 | 269.5 ± 16.0 | 113.8 ± 9.6 | 61.1 ± 6.6 | 0.0 ± 0.0 |
| PORCINE BIOPROSTHETICS | | | | | | |
| Glutaraldehyde treated | | | | | | |
| Stented Leaflet MAC2 | 173.4 ± 5.3 | 188.4 ± 4.8 | 123.1 ± 9.9 | 7.7 ± 1.6 | 0.0 ± 0.0 | 0.0 ± 0.0 |
| Stentless Leaflet MAC2 | 116.5 ± 7.3 | 90.2 ± 3.1 | 71.7 ± 4.1 | 11.9 ± 2.0 | 0.0 ± 0.0 | 0.0 ± 0.0 |
| Stentless Sinus MAC2 | 124.3 ± 10.3 | 99.5 ± 6.3 | 80.5 ± 4.2 | 13.4 ± 2.0 | 0.0 ± 0.0 | 0.0 ± 0.0 |
| Stentless Leaflet MAC3 | 131.4 ± 3.4 | 103.6 ± 3.0 | 80.2 ± 2.3 | 8.9 ± 0.7 | 0.0 ± 0.0 | 0.0 ± 0.0 |
| Stentless Sinus MAC3 | 140.7 ± 3.6 | 116.4 ± 2.3 | 90.2 ± 1.9 | 11.1 ± 0.8 | 0.0 ± 0.0 | 0.0 ± 0.0 |
| INERT MATERIALS | | | | | | |
| PTFE | 89.2 ± 2.4 | 9.9 ± 0.8 | 0.0 ± 0.0 | 6.8 ± 0.9 | 0.0 ± 0.0 | 0.0 ± 0.0 |
| Nitinol | 90.6 ± 2.0 | 19.3 ± 1.9 | 0.0 ± 0.0 | 8.0 ± 0.7 | 0.0 ± 0.0 | 0.0 ± 0.0 |

### Specific Cytokine Expression

### TNF-α

TNF-α titers for all test and control samples at each timepoint are tabulated in Table 3 and displayed in FIG. 7. At each timepoint, both the decellularized leaflet and sinus wall components of the aortic valves had significantly lower TNF-α responses as compared to freeze-fractured (P≦0.05) and native tissues (P≦0.05) within each species. The inert controls, the human decellularized and the glutaraldehyde crosslinked porcine biomaterials provoked the lowest TNF-α production. However, unlike the decellularized xenogeneic tissues, the TNF-α response to decellularized human was very low and fell towards negligible at later timepoints. There was prolonged elevation of TNF-α for the xenogenic tissues. While native human tissues provoked lower expression than native ovine or porcine, the freeze-fracturing treatment elevated the response for all three suggesting that with intact or fragmented cellular material, increased antigen recognition indeed was present.

### TGF-β1

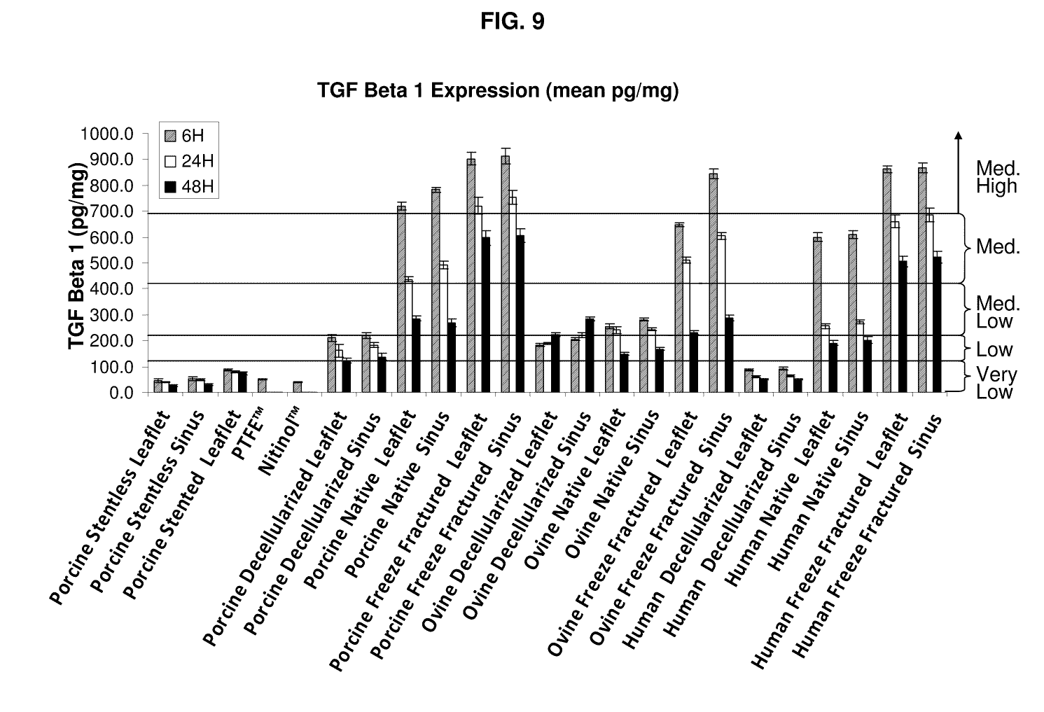

TGF-β1 titers were significantly lower for the decellularized tissues (Table 3 and FIG. 9), and most notably for human decell versus native (P≦0.05) and freeze-fractured (P≦0.05). In contrast, the TGF-β1 responses at later timepoints were elevated for the xenogenic tissues suggesting ongoing stimulation, perhaps reflecting the additional signaling functions of TGF-β1, which include wound healing and inflammatory amplification. While the porcine and ovine decellularized tissues were similar, the presence of cells (either freeze fractured or native) tended to result in higher titers for the porcine tissues as compared to ovine. FIG. 9 illustrates T6F-β1 titers at all three times for all materials (sinus wall and leaflets). As shown therein, the very low stimulation provoked by human decellularized tissues was similar to inert and glutaraldehyde crosslinked tissues and materials. Notably, T6F-β1 signaling was similar to the xenogeneic tissues, suggesting prolonged innate immune and more aggressive wound healing responses. Decellularization of the human tissues eliminated the T6F-β1 responses at all three time points as compared to native human and decell human (*P<0.05 as compared to native tissue from some species at same times; +P<0.05 as compared to PTFE and nitinol at same times). In this figure, the left vertical axis is pg cytokine/mg test tissue; the right vertical axis is standardized cytokine expression; and the error bars=±I.S.D.

### IL-6

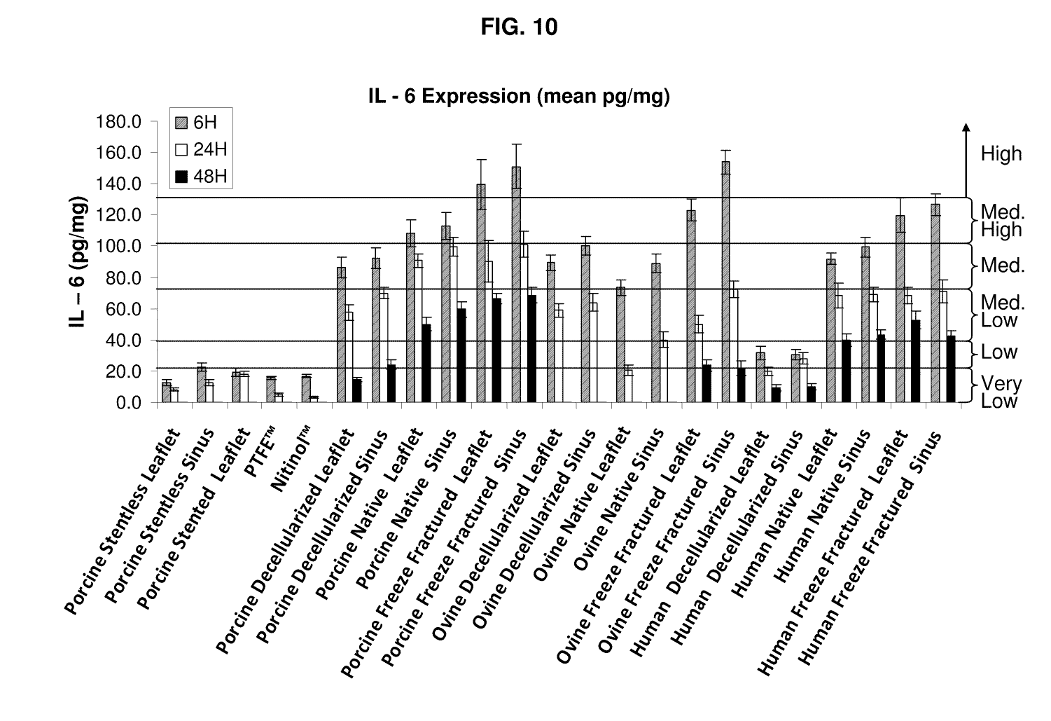

IL-6 expression was relatively short-lived for all test samples, always being maximal at the six-hour timepoint for each tissue preparation. (Table 3, FIG. 10). Decellularized human leaflets provoked a medium low response at six hours and very low responses thereafter but significantly less than the native or (P≦0.05) freeze-fractured human leaflets (P≦0.05). The uncrosslinked xenograft materials provoked higher IL-6 titers than did human. Inert and glutaraldehyde treated materials had the lowest and briefest expression. FIG. 10 illustrates IL-6 titers at all three times for all materials. Very low stimulation levels were provoked by the glutaraldehyde treated materials, PTFE and nitinol. The 6 hour levels for the human decellularized barely edge into the low expression range. The glutaraldehyde treated porcine, nitinol, PTFE and decellularized human provoked the briefest expression of IL-6. Decelluarization did not eliminate or significantly reduce IC-6 signaling provoked by the xenogeneic tissues as compared to their respective native unmodified tissue. (*P≦0.05 as compared to native tissue from same species at same times; +P<0.05 as compared to PTFE at same times). In this figure, the left vertical axis is pg cytokine/mg test tissue; the right vertical axis is standardized cytokine expression; and the error bars=±I.S.D.

### IL-2

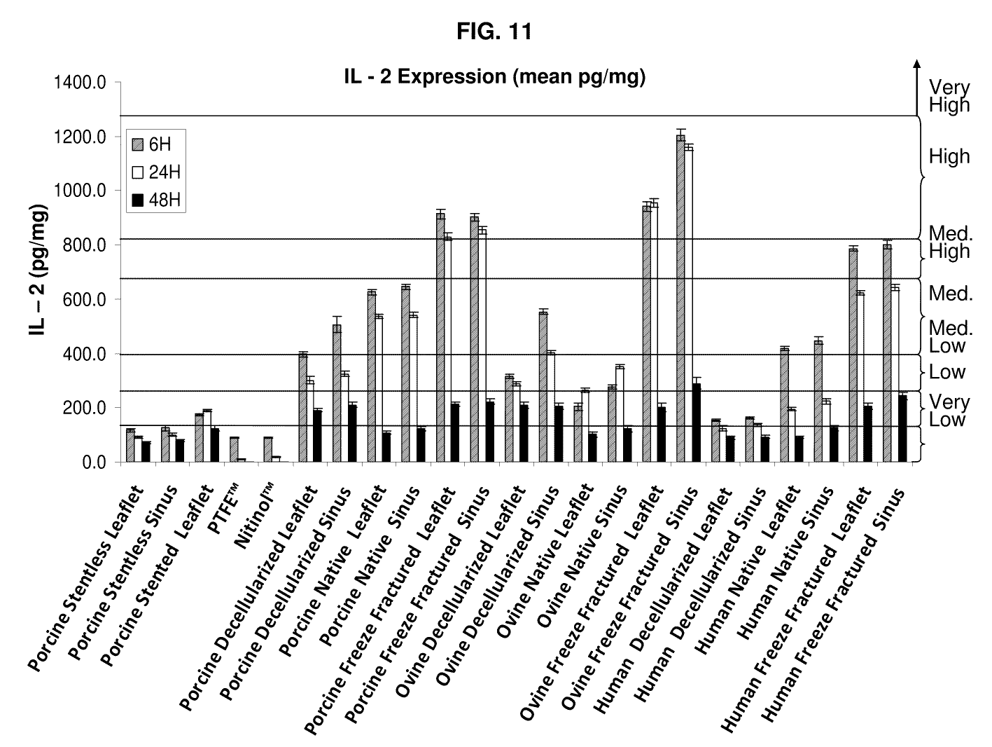

IL-2 expression was minimally provoked by human decellularized tissues but was markedly stimulated by the native and freeze-fractured human and by all uncrosslinked ovine and porcine tissues. (Table 3 and FIG. 11) The glutaraldehyde crosslinked bioprosthetic materials and the inert controls were again low stimulators although glutaraldehyde did not quite completely blunt the porcine bioprosthetics as compared to the inert materials. FIG. 11: IL-2 titers at all three time points for all materials tested. Human decellularized, PTFE, nitinol, porcine glutaraldehyde treated test tissues all remained at or below the boundary between low and very low expression. IL-2 expression fell off rapidly for all samples after 48 hours consistent with the early signaling role for this cytokine (*P<0.05 as compared to native tissue from same species at same time points; +P<0.05 as compared to PTFE at same time points). Left vertical axis pg cytokine/mg test tissue. Right vertical axis standardized cytokine expression. Error bars=±I.S.D.

### IL-1β-1 Expression

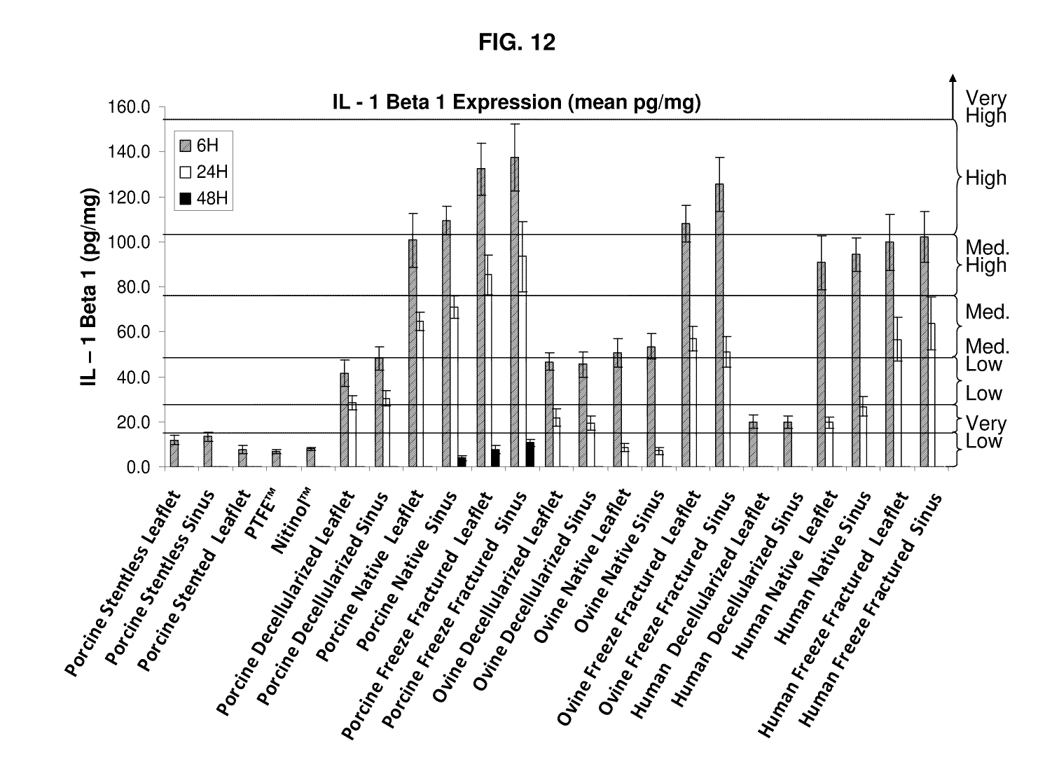

With profiles somewhat similar to the IL-6 expression, the IL-1β-1 production was, as expected, relatively short lived and minimal for the decellularized (especially human), and the crosslinked materials (Table 3, FIG. 12). The freeze-fractured xenogenic materials provoked the highest responses; porcine trended higher than ovine. Inert materials provoked negligible IL-β1 Expression. FIG. 12 illustrates IL-1β1 titers at all three time points for all materials tested. Human decellularized provoked low expression at 6 hours but rapidly fell to zero, whereas the PTFE, nitinol, and glutaraldehyde treated tissues expressed at very low levels at 6 hours then fell to zero. IL-1β1 was the briefest cytokine expression documented (as expected). Porcine native and freeze fractured seem to elicit higher responses than ovine native and freeze fractured, especially at the later time points (24 hours, 48 hours). (*P<0.05 as compared to native tissue from some species at same time points; +P<0.05 as compared to PTFE at same time points). In FIG. 12, the left vertical axis is pg cytokine/mg test tissue; the right vertical axis is standardized cytokine expression; and the error bars=±I.S.D.

### Multi-Cytokine Time Dependent Expression Profiles

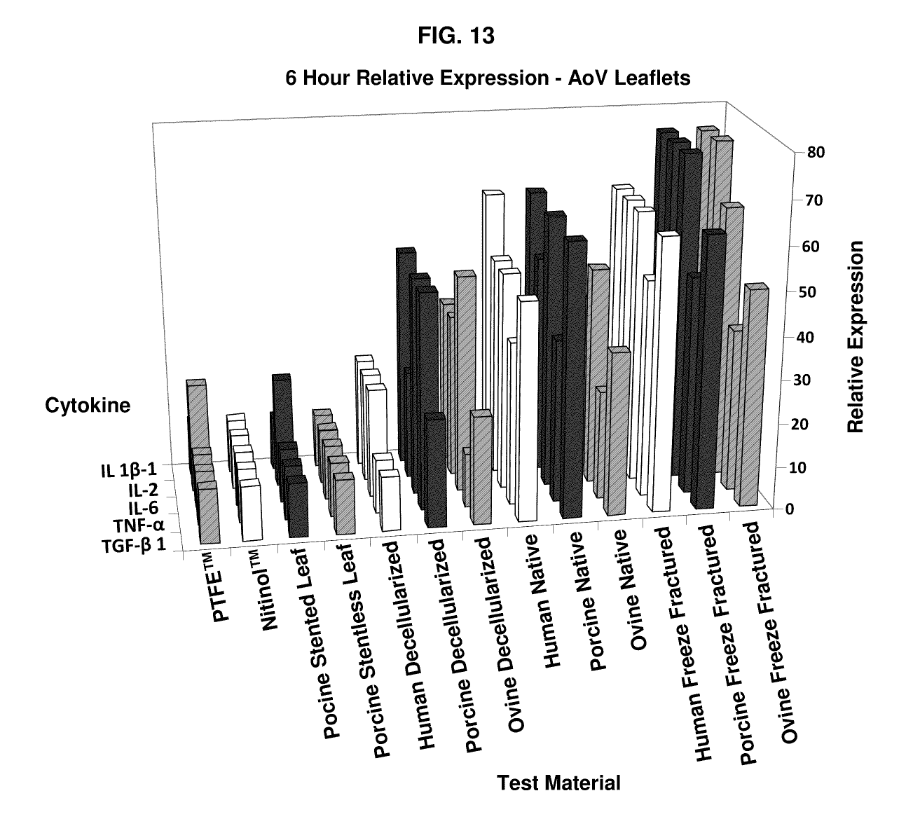

It is easier to visualize the time dependent expression with transformation of the absolute cytokine values to cytokine specific ordinal expression levels depicted as three dimensional profiles for each different material at each sampling time. (FIGS. 13-16). For example, the similarity of the 6, 24, and 48 hour profiles for the decellularized human tissue to the inert and crosslinked controls is readily apparent in these figures. In contrast, the human freeze-fractured and all xenogenic tissues elevated titers provoke higher, earlier, and with a slower decay. Each cytokine has a specific time course consistent with their putative signaling roles (eg, IL-1B-1 rapidly disappears, whereas TGF-B1 remains elevated). FIG. 13 illustrates relative cytokine expression by human macrophages after six hours of exposure to test materials (only controls and leaflets displayed for clarity). In FIG. 13, the Z axis=cytokines measured; the Y axis=relative expression; and the X axis=leaflets and control materials tested at this time point.

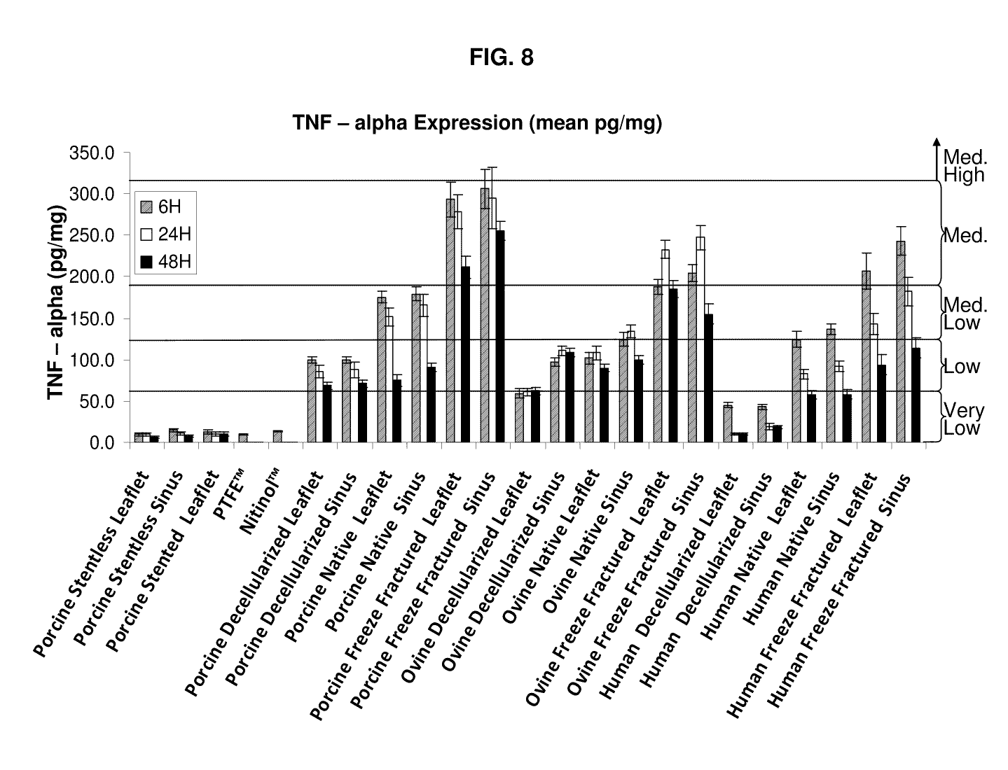

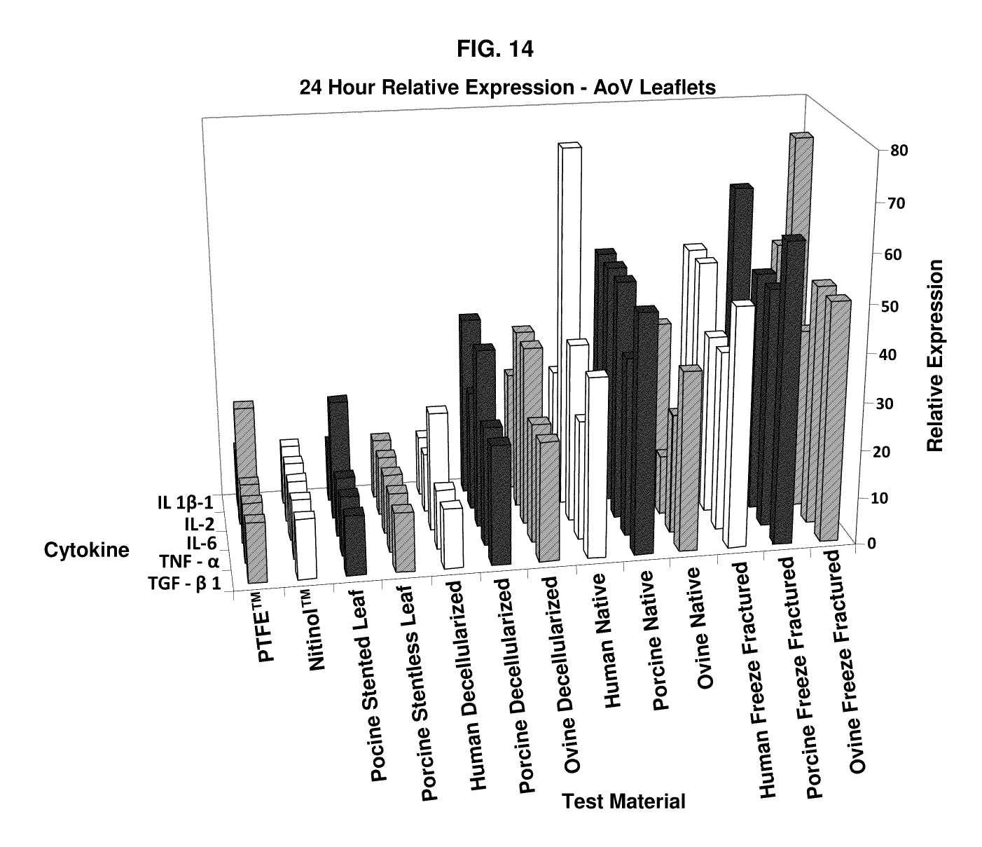

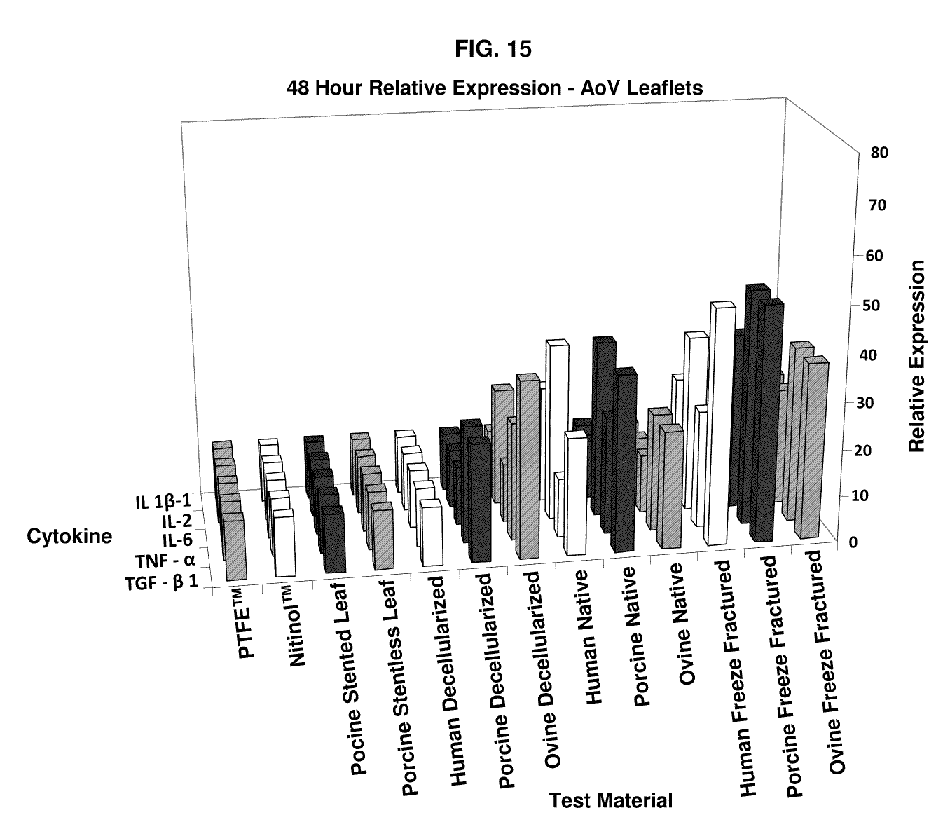

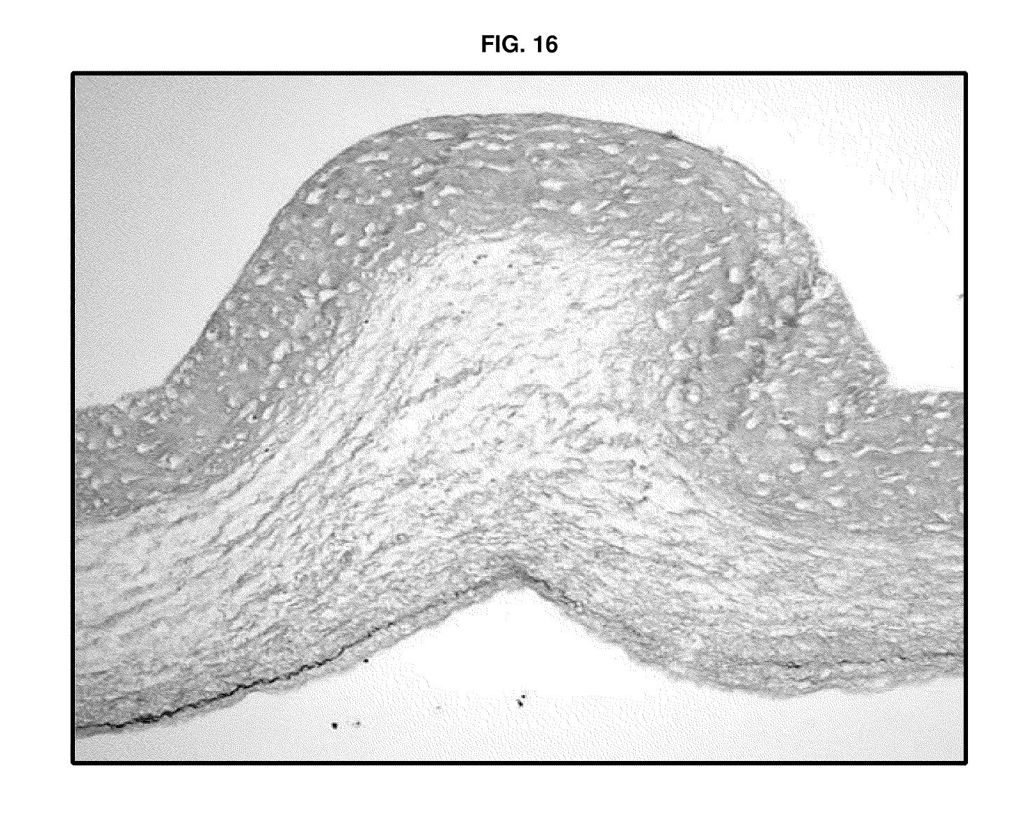

At six hours, the freeze-fractured materials, as expected, had the highest stimulated expressions followed by the native. (FIG. 14) The most benign profiles were recorded for decellularized and “inert” nonbiologic materials (PTFE and nitinol) and the glutaraldehyde crosslinked porcine bioprosthetic valve samples (FIGS. 14-16). Decellularized tissues had profiles similar to the glutaraldehyde crosslinked ECM scaffolds except for an early low level burst of IL-β1 (FIG. 14) and slightly higher levels of TNF-α and IL-6 at 24 hours (FIG. 15) and 48 hours (FIG. 16). Except for IL-1β1, the relative expression of all the cytokines remained elevated at 48 hours for freeze-fractured materials as compared to decellularized within species suggesting prolonged inflammatory stimulation by these antigen-rich materials. For all uncrosslinked xenogenic materials as compared to human, there were particularly robust TNF-α responses (FIGS. 8, 14, 15, 16). FIG. 8 illustrates the TNF-α titers at all three times for all materials tested. As shown therein, very low stimulation levels were provoked by the glutaraldehyde treated prosthetic materials, PTFE, nitinol and decellularized human valve tissues. Xenogeneic tissues provoked higher and the most prolonged TNF-α signaling. Freeze-fractured (Frz/Fx) tissues with disrupted cells expressed higher levels with less fall-off by 48 hours. In contrast to human valves, decellularization reduced but did not eliminate macrophage TNF-α signaling provided by the xenogeneic tissues (*P<0.05 as compared to native tissue from some species at same times; +P<0.05 as compared to PTFE at same times). FIG. 14 illustrates relative cytokine expression by human macrophages after 24 hours of exposure to test materials (only controls and leaflets displayed for clarity). The Z axis=cytokines measured; the Y axis=relative expression; and the X axis=leaflets and control materials tested at this time point. FIG. 15 illustrates relative cytokine expression by human macrophages after 48 hours of exposure to test materials (only controls and leaflets displayed for clarity). The Z axis=cytokines measured; the Y axis=relative expression; and the X axis=leaflets and control materials tested at this time point.

### Discussion

This study was designed to establish a quantitative bio-assay method for evaluating the inflammatory potential of putative ECM scaffolds for cardiovascular tissue engineering. Intentionally, the focus was on acute phase human macrophage-centric inflammatory cytokine signaling, when the presence of a foreign body would be initially detected. Since open heart surgery is itself a proinflammatory event, using monocytes transformed to macrophages and primed for inflammatory provocation is an appealing experimental design. These data confirm the original central hypothesis that decellularization of semilunar heart valves reduces inflammatory signaling (likely by the putative mechanism of antigen reduction). In contrast, the experimental model for abusive processing (freeze-fracture resulting in retained necrotic cell debris within the tissue) clearly potentiated recognition and cytokine signals at various steps of the inflammatory sequence. Additional insights were also revealed by the experiments. It is especially noteworthy given the long clinical experience, that glutaraldehyde seems to effectively mask antigen recognition. In contrast, unprocessed xenogenic tissues exhibited enhanced cross-species sensitization—not just by cells, but presumably by protein or carbohydrate xeno-antigens present in acellular ECM to which human macrophages may respond aggressively.

### Clinical Correlations to Inflammatory Potential of Bioprosthetic and Biological Heart Valves

The historical transition from formaldehyde to glutaraldehyde fixation for the manufacture of crosslinked porcine xenograft valve prostheses for clinical use was certainly fortuitous, given our demonstrated suppression of cytokine signaling by glutaraldehyde. Although some nickel alloys have been demonstrated to promote cytokine expression, the tested control nitinol was a very low stimulator, consistent with clinical experience. PTFE is a nondegradable polymer which has long and salubrious clinical experience in surgical cardiovascular reconstructions, and tested well in this bench assay. Such implantable materials are felt to provoke minimal inflammatory response and typically are described as eliciting benign foreign body responses of the innate immune system related to local wound healing. This “benign inflammatory response” can be defined by characteristic quantitative cytokine signaling profiles. The current limited clinical durability of “viable” cryopreserved “homograft” heart valves demonstrates that despite numerous positive surgical attributes, there are limitations to implanting inherently proinflammatory materials as exemplified by our native and freeze-fractured test groups.

Mechanistic theories explaining the limited clinical durability of crosslinked xenograft bioprosthetic heart valves have suggested sequences related to physical and chemical deterioration leading to calcium accumulation and failure. More recent evidence suggests an important role for inflammation and immune mechanisms modulated by recipient factors such as age, immune competency, treatment with anti-rejection medications, etc., as the modulators of bioprosthetic heart valve fibrocalcific degeneration, and may be a similar process to the pathogenesis of degenerative native valve disease and atherosclerosis. However, as the implant duration increases glutaraldehyde leaching may uncover antigen sites, potentiating the inflammatory response, resulting in leaflet calcification and structural deterioration. This is especially accelerated and aggressive in younger patients with robust immune responses. The data are consistent with a significant role for this hypothesized masking and unmasking of antigen sites.

Allograft and xenograft semilunar valves are attractive as scaffolds for bioengineered valves for many reasons. The documented early clinical failures of incompletely decellularized xenograft tissue valve are also consistent with the findings in this study and suggest mechanisms that explain why porcine ECM continues to be proinflammatory when implanted into humans. When decellularization is incomplete, results seem to be worse, even with allograft tissues. The extensive clinical experience with the variable durability for cryopreserved homograft valves that variably contain process dependent residual cells that are viable (somewhat proinflammatory), necrotic (very proinflammatory) and apoptotic (non-inflammatory) is consistent with at least a semi-quantitative relationship between antigen provocation, inflammatory signaling, and bioprosthetic valve failure. Within species, decellularization does appear to “de-antigenize” heart valves although it does not necessarily preclude minimal wound healing type inflammation as even implants of benign “inert” materials will provoke a brief recognition marked by slight macrophage signaling as we documented for nitinol and PTFE. Functional results at four to five years with the only currently commercially available decellularized heart valve in the pulmonary position are promising but have not yet decisively demonstrated improvement relative to standard cryopreserved. This may reflect a spectrum of decellularization efficacy, substrate biological variability, patient specific factors (eg, age) or that ultimate benefit is only to be seen in the longer term results. Conversely, animal studies suggest that allograft decellularization reduces calcification rates, prolongs durability and improves performance when tested in the classic and robust juvenile sheep model. Decellularized valves have been tried in very limited trials as scaffolds for cell seeded tissue engineered valves with early encouraging results.

### Macrophage Cytokine Signaling and Selection/Optimization of Candidate Biomaterials for Scaffolds

The quest for a non crosslinked biological semilunar heart valve of either xenogeneic or allogeneic origins has been highly instructive, beginning with the variable efficacy of various decellularization protocols. Various endpoints such as residual DNA content, histological evidence of residual cells, etc. are useful for gauging process efficiency, but perhaps the more pertinent endpoint is when the candidate tissue has been rendered minimally proinflammatory.

The fabrication of scaffolds for tissue engineering heart valves is subject to multiple processing and engineering variables beginning with the selection of the underlying material such as a polymer, ECM-derived, and polymer/ECM hybrids. If an ECM scaffold composition is chosen, it can be theoretically derived from xenograft material, allograft material or totally synthetic constructs. Macrophage signaling data could predict the clinical experience which, for example, in the case of unmodified xenograft ECM, suggests that it would be a poor choice risking accelerated rejection, inflammation, degradation and deterioration of tissue functionality. Alternatively, conceptually once a substrate is selected, various “conditioning” treatments could be applied to enhance cell adhesion, migration and differentiation, as well as to reduce inflammation, minimize calcification, enhance wound healing, improve rheologic performance, or other critical parameters. When not specifically designed or demonstrated to reduce innate immune responses each “treatment” to enhance various performance parameters has itself the risk of unintentionally introducing proinflammatory characteristics for which appropriate testing should be done to exclude such consequences. This approach is already being used with manufactured xenogeneic valves that employ “anticalcification” treatments and rely on glutaraldehyde to camouflage antigen sites.

### Xenogeneic Biomaterials

The muted inflammatory macrophage mediated cytokine profiles evoked by crosslinked porcine prosthetic valve leaflets were striking and suggest that despite xenogeneic origin, the initial short to medium term satisfactory clinical experience with these bioprostheses may be, in part, due to inflammatory signaling delayed or suppressed by the glutaraldehyde. In contrast, the unfixed porcine and ovine tissues were much more provocative. These data suggest highly significant cross species antigenicity. Rieder and colleagues in Vienna, Austria, have demonstrated that porcine decellularized valves stimulate enhanced human monocyte homing. While exploring a somewhat different macrophage function, their data are supportive of our findings indicating that xenograft structural proteins, in the absence of masking by methods such as glutaraldehyde crosslinking, can elicit an enhanced inflammatory response. Structural moieties such as collagen and elastin have traditionally been felt to be genetically generally conserved across mammalian species. However, there are known specific epitope exceptions such as the potent carbohydrate xenoantigen α-gal which is not expressed in humans and other Old World primates. Uncrosslinked ovine and porcine tissues tested poorly as compared to human, which is consistent with the theory that antigens other than HLA or ABO related, such as carbohydrate xenoantigens (eg, α-galactosyl epitope) may play a critical role. Even if not due specifically to α-gal discordancy, our results suggest that using unmodified porcine or ovine valve tissue (even when decellularized) as scaffold material for tissue engineering structures for clinical human implants may be hazardous. Conversely, these lower mammals might be too evolutionarily distant from hominoids to provide valid in vivo milieu for direct testing of acellular human tissues.

### Rationale for the Choice of the Specific Cytokine Targets for Assessing Inflammatory Signaling Responses

There are numerous inflammatory cytokines and chemokines with pleiotropic, overlapping and intricately related functions. Given the goal of establishing a quantitative bench assay that could predict the proinflammatory characteristics of putative clinical biomaterials, we selected a tractable set of cytokines with varying roles but with a bias towards early phase critical signals. TNF-α is a potent, acute phase, local and systemic, and perhaps the critical proinflammatory signaling cytokine that activates NFkβ and MAPK pathways and functions in paracrine, juxtacrine and autocrine fashions. IL-2 induces proliferation of T-Lymphocytes. IL-1-β-1 is an early acute phase responder that activates and recruits macrophages, is synergistic with TNF-α, and promotes synthesis of acute phase hepatic proteins, pro-coagulants and scar tissue proteins. IL-6 is typically a bit more downstream (stimulated by the very early activation of IL-1-β1) and has endocrine functionality. IL-6 has also been linked to trauma, foreign body responses, tissue damage inflammation as well as being a known vascular smooth muscle proinflammatory cytokine implicated in atherosclerosis, coronary stent stenosis, and degenerative valve disease. TGF-β1 is a member of the TGF-β family and in the context of inflammation, wound healing, fibrosis and calcification, is a particularly complex and multifaceted moiety with a panoply of roles, interdigitating with numerous acute and chronic signaling pathways, some of which are beneficial and others contribute to dystrophic responses. The TGFβ-BMP pathway has been implicated in the fibrocalcific degeneration of heart valves, which supports the mechanistic theory incriminating a subacute chronic inflammatory process. Cytokine proteonomic profiles following challenge, have characteristic time dependent expression, as demonstrated in our study. Anti-inflammatory cytokines such as IL-10 may concomitantly gradually increase with time suggesting that the resolution (or the lack thereof) of foreign body inflammatory responses have multiple cytokine effectors.

Limitations of this Study

Material or stress related inflammatory mechanisms unrelated to antigenicity (eg, elevated leaflet residual stresses or strains, collagen bundle fracture) also contribute to the progressive loss of durability of crosslinked porcine bioprosthetic heart valves. Many of the currently implanted bioprosthetic valves have been designed to reduce the residual tissue stresses resulting in a reduction in structural deterioration. As a static assay, our method might not measure the benefits of such biomechanical effects of processing. Some prosthetics have been treated with anticalcification agents that slow the mineralization of calcium without necessarily altering the stimulatory elements (ie, a downstream treatment to mitigate the consequences of inflammation). By design, our assay does not account for additional macrophage, or leukocyte recruitment, thus this assay does not precisely mimic the in vivo milieu in which continued resident tissue macrophage recruitment, circulating monocyte homing, and cell-cell interactions amplify and modify the cell signaling cascade. For example, immune specific responses are enhanced by lymphocyte participation. However, the goal of these studies was to explore an in vitro assay method that would measure early phase events as predictors of in vivo performance. The profiles defined by these studies are consistent with the clinical experience for these materials.

### Implications for Tissue Engineering Clinically Useful Cardiac Valves

These current results suggest that when decellularization of human valve scaffolds is essentially complete, there is significant reduction of inflammatory cytokine signaling. From this perspective, it seems attractive to base a putative clinical TEHV on decellularized human ECM scaffolds derived from cryopreserved heart valves acquired during multiorgan and tissue harvests, transported, screened and prepared in AATB accredited tissue processing banks, rather than using the albeit more convenient xenogenic foodstock animal (eg, ovine or porcine) sources. To achieve consistency as platforms for tissue engineered replacement heart valves (TEHV), it should be useful to have definable criteria for the relative inflammatory potential of putative scaffold materials. Our assay could also be used to assess processing efficacy for specific decellularization protocols. Such testing could be extended to any proposed implant material. It appears that much of the defining high responder information is present at six hours. By adopting multiplex technology rather than the classical ELISA methods we employed, this assay could be modified into a very efficient high throughput screening method. With that approach many additional chemokines and cytokines could also be assayed to identify optimal response profiles. The longer, more complex in vivo wound healing assays could then be performed on identified low response candidate materials to further explore inflammation characteristics. Alternatively, when the high inflammatory actors are known, direct measurement of the culprit antigens could be done with standard immunoblotting techniques.

The current interest in tissue engineering heart valves is based on the concept that a carefully selected “nonreactive” protein ECM valve scaffold might achieve prolonged protection from dysfunctional deterioration by active participation in the matrix protein degradation-resynthesis cycle by seeding autologous valve interstitial cells capable of continuing ECM protein degradation/resynthesis cycles, thus providing the appropriate substrate and the means for both constructive and adaptive remodeling. The presence of a functional myofibroblast valve interstitial cell population within a non-provocative scaffold should provide a useful engineered construct for surface endothelial cell repopulation, the presence of which would diminish prothrombotic inflammatory provocation, particularly beneficial since the lumenal surfaces of such tissue engineered valves would be exposed to both the immunobiology and the mechanical stresses (eg, shear) of the circulation. Conversely, a proinflammatory scaffold may negate the beneficial effect of cell seeding or even result in scar formation rather than salubrious healing and tissue regeneration.

That even the most benign materials elicited measurable, albeit low level cytokine expression, is consistent with the essential surveillance, wound healing and regenerative functions of macrophages. More recently, tissue based macrophage activation in vivo has been explored as a diverse spectrum of polarized phenotypes in which the “M1” macrophage profile describes pro inflammatory anti-pathogen responses while “M2” macrophages promote immuno-modulatory, tissue repair and remodeling. The relevance of this taxonomy to our cell based assay is not clear. We did not measure the defining surface markers (eg, CD 168 and CCR7), but since LPS activation was used, the sensitized macrophage population for this assay was primed for production of inflammatory cytokines. Uniquely, implanted valved conduits are exposed on the advential side to vascularized granulation tissue ingrowth and on the lumenal side to the circulation. Thus these constructs are presented immediately upon implantation to both circulating and tissue based immune mechanisms.

Currently approved clinical biological/bioprosthetic heart valve replacement options (allograft and xenografts) exhibit limited post implant durability (likely due to innate inflammation and immune rejection and consequential calcification), ultimately leading to accelerated failure. Cryopreserved “viable” (ie, containing donor cells) homografts as currently clinically used are known to have limited durability due to inflammation and immune rejection resulting in fibrosis and calcification of the implanted valves resulting in valvular stenosis and/or insufficiency. It has been demonstrated as a result of this investigation that efficient decellularization can remove macrophage provocative elements from donor allograft valves perhaps providing antigen-reduced collagen and elastin extracellular matrix (ECM) scaffold that retains optimal structural elements of normal semilunar valves. HLA antigenic debris are absent following adequate decellularization of valves and in the available studies have blunted post implant panel reactive antibody titers.

Decellularized valves are attractive clinically as they surgically can be tailored for size, location and functional performance. These valves achieve normal immediate function post implantation and in the absence of traditional crosslinking, the proteins are available for resynthesis, remodeling and perhaps growth, and thus may have the potential for prolonged durability. However, these data suggest that non-human valve tissues, even when decellularized, retain proinflammatory characteristics and are perhaps a risky choice for an acellular ECM scaffold for clinical applications. If, as predicted by the cytokine expression profile assays, decellularized human allograft ECM scaffolds are minimally proinflammatory in vivo, subject only to benign wound-healing, then these may be highly suitable substrates either as implantable acellular constructs or as scaffolds with which to assemble tissue engineered viable personal heart valves (TEHV's) using ex vivo bioreactor based cell seeding strategies and/or in vivo directed autologous recellularization.

### Example 4

This example illustrates the preferred decellularization process.

### Materials and Methods

### Solutions Used:

a. Triton X-100 (Triton): 0.05% Triton X-100 solution a 1:2000 dilution derived from 100% Triton X-100 detergent (Sigma T8787) in ddH₂O. 200 mL needed per valve. Can be made ahead of time.
   For 2 L use 1 mL 100% Triton-X, 1999 mL ddH₂O.
b. N-lauroylsarcosine Sodium Salt Solution (NLS): 1% NLS Solution a 1:20 dilution derived from 20% Sodium Laureth Sulfate (Sigma-L7414) in ddH₂O. 200 mL needed per valve. Can be made ahead of time.
   For 2 L use 100 mL 20% NLS, 1900 mL ddH₂O
c. Hypertonic Salt Solution (HSS): 1% NaCl (Fisher-BP358-1), 12.5% D-Mannitol (Sigma-M9647), 5 mM MgCl₂ (Sigma-M2643), 500 mM KCl (Sigma P4504) in NS (Normal Saline). Can be made ahead of time. 200 mL per valve needed.
   For 2 L use 2 L NS, 18 gm NaCl, 2.03 gm MgCl₂, 74.3 gm KCl, 250 gm Mannitol.
d. 2×Saline Mannitol Solution (SMS): 1% NaCl (Fisher-BP358-1), 12.5% D-Mannitol (Sigma-M9647). 200 mL needed per valve needed. Can be made ahead of time.
   For 2 L use 2 L NS, 18 gm NaCl, 250 gm Mannitol.
e. RNA-DNA Enzyme Extraction Buffer (BENZ): 12.5 KU of Benzonase® (Sigma-E1014) per 200 mL ddH₂O, 8 mM MgCl₂ (Sigma-M2643), pH to 8.0 using diluted NH₄OH (˜100 μL needed of 1M solution). Should be made the day of use. 400 mL needed per valve.
   For 400 mL use 400 mL ddH₂O, 1 vial Benzonase® (25 KU), 650 mg MgCl2 (Sigma-M2643)
f. Organic Solvent Extraction Buffer (EtOH): 2:5 dilution of ethyl alcohol 200 proof (Sigma-459836) in ddH₂O—40% v/v solution. Can be made ahead of time. 200 mL needed per valve.
   For 2 L use 800 mL ethanol, 1200 mL ddH₂O

Valves were dissected in a laminar flow safety cabinet using sterile technique and stored individually, in 200 mL of preprocessing storage solution in sterile 250 mL jars for 72 hours at 4° C.

On Day One of processing the detergent and osmotic shock sequences were performed. The 250 mL flasks containing the valve tissue were each filled with 200 mL HSS with one heart valve in each jar. Flasks were then placed on a rocker plate for 2 hours at 220 RPM at RT. The valves were then washed for 3 hours in Triton at 220 RMP at RT at a temperature of 21° C. Each wash or rinse was conducted in a new sterile 250 mL flask and transfer was completed under a sterile laminar flow hood. A rinse was then performed on the valves one time for 10 minutes in ddH₂O at 220 RPM at RT. The valves were then washed for 2 hours in HSS at 220 RPM at RT. Another rinse was performed for 1 hour in ddH₂O at 220 RPM at RT. The valves were then washed for 3 hours in Triton at 220 RPM at RT. Next, a RNA-DNA enzyme extraction was performed. A flask containing sterilized BENZ at a pH of 8.0 was used for the extraction and the valves were transferred into the BENZ solution to shake O/N on a rocker plate at 220 RPM at 37° C. overnight.

On Day Two of Processing, the valves were risked for 1 hour in ddH₂O at 220 RPM at RT, washed, and then placed in NLS solution on a rocker plate O/N at 220 RPM at RT.

On Day Three of Processing, an organic extraction was performed. Valves were rinsed once for 4 hours in ddH₂O at 50 RPM at RT. Next, and extraction was completed using ethyl alcohol. For the extraction, the valves were rinsed for 30 minutes with 40% EtOH at 50 RPM at RT. After the extraction, an ion exchange detergent residual extraction for dual chamber was set up. FIG. 1 illustrates how the exchange chamber was assembled. 50 gm of each type of bead were used. The beads were soaked in EtOH for 5 minutes and then quickly rinsed in ddH₂O. The beads were then aseptically added to and 8 L spinner flask. The valves were then aseptically added to the 10 L bioreactor flask. Throughout this process, all connections were sprayed down with 70% EtOH as needed. The spinner flasks were then filled with 7 L ddH₂O by connection ports to 10 L reservoir via peristaltic pump and silicone tubing. Both stir plates were spun at 60 RPM and the peristaltic pump was set to 48 RPM (150 mL/min, max. setting).

On Day Four of Processing, a Mannitol soak was performed. The Soak was carried out for those valves which were not immediately being placed into the post-decellularization storage solution for immediate use. For those valves placed in the soak, they were soaked for 2 hours in 200 mL SMS on a rocker plate at 50 at RT. A new sterile 250 mL flask with 200 mL post-decellularization storage solution was used to place each valve in for storage purposes.

1. A tissue engineered heart valve comprising a previously decellularized tissue that has undergone a cell seeding process wherein said tissue engineered heart valve is characterized by having at least 20% of the cells that remain on or in said previously decellularized tissue two weeks after the cell seeding process are located below or interior to the basement membrane of said tissue.

2. The tissue engineered heart valve of claim 1, wherein at least 50% of the cells that remain on or in said previously decellularized tissue two weeks after the cell seeding process are located below or interior to the basement membrane of said tissue.

3. The tissue engineered heart valve of claim 1, wherein at least 80% of the cells that remain on or in said previously decellularized tissue two weeks after the cell seeding process are located below or interior to the basement membrane of said tissue.

4. The tissue engineered heart valve of claim 1, wherein said heart valve has a low or very low inflammatory response as measured by the expression of cytokines.

5. The tissue engineered heart valve of claim 4, wherein said cytokines are selected from the group consisting of TNF-α, TGF-1-β, IL-6, IL-2, IL-1-β-1, and combinations thereof.

6. The tissue engineered heart valve of claim 1, wherein said tissue engineered heart valve comprises a harvested allogenic tissue that has been decellularized and recellularized.

7. The tissue engineered heart valve of claim 6, wherein said recellularization is completed in an environment with the presence of cyclic pressure.

8. The tissue engineered heart valve of claim 7, wherein said environment is a bioreactor.

9. A bioengineered construct prepared by a method comprising the steps of: reciprocating osmotic shock sequences, a detergent wash, a second reciprocating osmotic shock sequence, a RNA-DNA extraction, a digestion step, an enzyme treatment, a second detergent step, an organic solvent extraction, an ion-exchange detergent residual extraction, and a final organic extraction.

10. The bioengineered construct of claim 9 wherein said construct is used as the decellularized tissue of claim 1

11. A method for recellularizing a heart valve comprising the steps of:
a. obtaining a decellularized heart valve;
b. introducing cells to said decellularized heart valve in an environment subject to cyclic pressure, wherein said cyclic pressure leads to pulsatile motion in said environment.

12. The method of claim 11, wherein said environment is a bioreactor.

13. The method of claim 11, wherein said cyclic pressure is between −5 mmHg to 30 mmHg.

14. The method of claim 11, wherein said cyclic pressure increases over time in a sinusoidal waveform motion.

15. The method of claim 16, wherein said cyclic pressure is increased at least two times.

16. The method of claim 15, wherein said cyclic pressure is increased at intervals of 48 hours or less.

17. The method of claim 11, wherein said cells comprise 2.4×10⁴ to 2.5×10⁷ cells.

18. The method of claim 12, wherein at least 20% of said cells migrate below the basement membrane of said heart valve at two weeks post introduction of cells.

19. A method of reducing the inflammatory response of a tissue comprising the step of decellularizing the tissue using a method comprising the steps of: reciprocating osmotic shock sequences, a detergent wash, a second reciprocating osmotic shock sequence, a RNA-DNA extraction, a digestion step, an enzyme treatment, a second detergent step, an organic solvent extraction, an ion-exchange detergent residual extraction, and a final organic extraction.

20. A tissue engineered heart valve comprising a decellularized heart valve that has been recellularized with autologous cells using a recellularization process, wherein said decellularized heart valve, prior to recellularization, is characterized by a low to very low inflammatory potential as measured by cytokine expression, and wherein at least 20% of the cells that remain on or in said previously decellularized tissue two weeks after the recellularization process are located below or interior to the basement membrane of said tissue.
